(19) United States
(12) Patent Application Publication
(10) Pub. No.: US 2023/0348847 A1
(43) Pub. Date: Nov. 2, 2023

(54) HYDROGEL ASSISTED STEREOLITHOGRAPHIC ELASTOMER PROTOTYPING

(71) Applicant: Washington University, St. Louis, MO (US)

(72) Inventors: Nathaniel Huebsch, St. Louis, MO (US); Daniel Simmons, St. Louis, MO (US); David Schuftan, St. Louis, MO (US); Guy Genin, St. Louis, MO (US); Ghiska Ramahdita, St. Louis, MO (US)

(21) Appl. No.: 18/140,367

(22) Filed: Apr. 27, 2023

Related U.S. Application Data
(60) Provisional application No. 63/335,804, filed on Apr. 28, 2022.

(51) Int. Cl. C12N 5/00; C12N 5/077; B33Y 10/00; B33Y 80/00

(52) U.S. Cl. CPC C12N 5/0062; C12N 5/0657; B33Y 10/00; B33Y 80/00; C12N 2513/00; C12N 2537/10; C12N 2533/30; C12N 2533/74; C12N 2533/76

(57) ABSTRACT

Disclosed herein are methods of making a mold for use with cells, such as engineered tissue. The method includes printing a 3D printed resin mold, casting a hydrogel over the 3D printed resin mold to create a crosslinked hydrogel negative mold, and casting a silicone rubber elastomer over the hydrogel negative mold to create a master mold.

### CROSS REFERENCE TO RELATED APPLICATIONS

This application claims the benefit under 35 U.S.C. § 119(e) to U.S. Provisional Application 63/335,804, filed Apr. 28, 2022, the entire contents of which are incorporated herein by reference in their entirety.

### GOVERNMENT SUPPORT

This invention was made with government support under HL107594 awarded by the National Institutes of Health, and CMMI1548571 awarded by the National Science Foundation. The government has certain rights in the invention.

### FIELD OF THE INVENTION

The present invention relates generally to hydrogel assisted stereolithographic elastomer prototyping.

### BACKGROUND

It is challenging to obtain complex, 3D shapes with fine features using traditional soft lithography. Soft lithography is a common technique for creating molds for engineered tissues at high density for medium throughput, but inherently limits the designs produced. This technique is time consuming, requires fabrication of the master mold in clean room facilities, and is limited to designs utilizing extrusions of a 2D feature. New techniques have been created in attempts to fabricate 3D shapes, but require enhanced training and further raise the barrier to entry in creating engineered tissues. Additionally, repeated use of the silicon-epoxy master mold can lead to mold destruction, requiring new molds to be created fairly frequently.

These inherent limitations with soft lithography have led to a desire to replace this fabrication method with 3D printing. 3D printing enables rapid design changes and production, creating shapes that are difficult or impossible using photolithography. However, the materials used for high resolutions inhibit silicone rubber elastomers like poly(dimethyl siloxane) (PDMS) crosslinking, leading to inadequate replication. Various methods have been attempted to enable ‘direct’ PDMS molding off 3D print. However, these methods are often inconsistent in their success and require specific timing, have been reported to be user dependent, or may subject the 3D print to conditions that occasionally lead to surface damage or deformation. Some methods require spraying 3D prints with a uniform coating of release agents, which are amenable to simple extruded surfaces but would be less successful for more complicated 3D shapes such as those with hooks or overhangs. This means 3D prints cannot be used to directly create PDMS molds with fine features.

Accordingly, there is a need for a method to allow replication of a 3D printed resin into silicone rubber elastomers.

### BRIEF SUMMARY

The disclosure provides for methods for replicating a 3D printed resin into PDMS, through an intermediate step using a hydrogel.

In some aspects, the method may include printing a 3D printed resin mold; casting a hydrogel over the 3D printed resin mold to create a crosslinked hydrogel negative mold; and casting a silicone rubber elastomer over the hydrogel negative mold to create a master mold.

In an aspect, the hydrogel may crosslink in a manner independent of the small molecules released by 3D printed resins, which are disruptive to elastomer crosslinking. The crosslinked hydrogel negative mold comprises a high crosslink density. The crosslink density may be greater than about 2.5 mol/cm³ or greater than about 4 mol/cm³. The crosslinked hydrogel negative mold may have a toughness of greater than 8 kJ/m³. For example, the hydrogel may be agar, alginate, hyaluron, or gelatin. In some examples, the hydrogel may include 1.5% w/v agar or may include 1 high molecular weight alginate and 2% low molecular weight alginate.

In an aspect, the silicone rubber elastomer is poly(dimethyl siloxane). The master mold may be a 1:1 replication of the 3D printed resin mold. The master mold may replicate features as small as 10 μm from the 3D printed resin mold. The master mold may not contain any leachate from the 3D printed resin mold. For example, the master mold may be biocompatible and may not contain any toxins that would inhibit cell growth, even if the material used for the 3D printing is toxic or produces toxic leachate.

The disclosure further provides for a master mold made using the disclosed method. For example, the master mold may include a silicone rubber elastomer and have a Procrustes score of greater than 0.99, representing excellent fidelity of replication, when compared to a 3D printed resin mold used to make the master mold. In addition, the master mold may not contain any leachate from the 3D printed resin mold.

The disclosure further provides a method for forming an engineered tissue using the disclosed method. For example, the method for forming an engineered tissue may comprise printing a 3D printed mold, casting a hydrogel over the 3D printed mold to create a crosslinked hydrogel negative mold, casting a silicon rubber elastomer over the crosslinked hydrogel negative mold to create a master mold, and seeding the master mold with cells to produce the engineered tissue. In some examples, the master mold may be placed in a tissue culture well plate prior to being seeded with cells.

In an aspect, the engineered tissue may comprise cardiomyocytes and stromal cells such as fibroblasts, or mesenchymal stem cells. In an example, the engineered tissue may display an action potential and a calcium transient. In a further example, the engineered tissue may be a model of an in vivo organ system.

Additional aspects and features are set forth in part in the description that follows, and will become apparent to those skilled in the art upon examination of the specification or may be learned by the practice of the disclosed subject matter. A further understanding of the nature and advantages of the disclosure may be realized by reference to the remaining portions of the specification and the drawings, which forms a part of this disclosure.

### DESCRIPTION OF THE DRAWINGS

The description will be more fully understood with reference to the following figures, which are presented as variations of the disclosure and should not be construed as a complete recitation of the scope of the disclosure, wherein:

FIG. 1 is an example of the disclosed method.

FIGS. 2A, 2B, 2C, 2D, 2E, 2F, and 2G are schematics describing the disclosed to replication 3D prints into a PDMS elastomer mold, in one example.

FIG. 3A is a representative image of PDMS directly molded off of SU-8 on silicon (soft lithography substrate). FIGS. 3B-3D are representative images of PDMS directly molded off of a high-resolution 3D print.

FIG. 4 is an example of qualitative scoring.

FIGS. 5A and 5B are graphs of qualitative or Procrustes scores for PDMS molds using agar or alginate negative molds at various crosslink concentrations.

FIGS. 6A and 6B are graphs of qualitative or Procrustes scores for PDMS molds using agar or alginate negative molds at various toughness's.

FIG. 7A is a representative CAD drawing of a geodesic dome 3D shape that is 3D printed. FIG. 7B is a scanning electron microscope image of the 3D printed shape. FIGS. 7C and 7D are a scanning electron microscope images of the 3D printed shapes replicated in PDMS that would be difficult or impossible to create using standard photolithography methods. Scale bars: 1 mm. FIG. 7E is a brightfield image of the PDMS mold of FIG. 7D.

FIG. 7A is a representative CAD drawing of a 3D shape that is 3D printed. FIGS. 7B and 7C are scanning electron microscope images of the 3D printed shapes replicated in PDMS that would be difficult or impossible to create using standard photolithography methods. Scale bars: 1 mm. FIG. 7D is a brightfield image of the PDMS mold of FIG. 7B.

FIG. 8A is a representative CAD drawing of a 3D shape that is 3D printed. FIGS. 8B and 8C are scanning electron microscope images of the 3D printed shapes replicated in PDMS that would be difficult or impossible to create using standard photolithography methods. Scale bars: 1 mm. FIG. 8D is a brightfield image of the PDMS mold of FIG. 8B.

FIG. 9A is a representative CAD drawing of a dog-bone 3D shape that is 3D printed. FIGS. 9B and 9C are scanning electron microscope images of the 3D printed shapes replicated in PDMS that would be difficult or impossible to create using standard photolithography methods. Scale bars: 1 mm. FIG. 9D is a brightfield image of the PDMS mold of FIG. 9B.

FIGS. 10A and 10B are example images of engineered tissue created using the PDMS mold made using the disclosed method.

FIGS. 11A and 11B are examples of crosslinking mechanisms for hydrogels.

FIGS. 12A-12C are examples of alginate hydrogels having only covalent bonds.

FIG. 13A is a scanning electron microscope image of a 3D printed shape.

FIGS. 13B-D are scanning electron microscope images of PDMS molds having different qualitative scores. Scale bars: 1 mm.

FIG. 14A is a graph of qualitative scores for PDMS molds using agar or alginate negative molds at various crosslink concentrations.

FIG. 14B is a graph of qualitative scores for PDMS molds using agar or alginate hydrogels at various toughness's.

FIG. 14C is a graph of qualitative scores for PDMS molds using agar or alginate hydrogels at various ultimate modulus's.

FIGS. 15A and 16A are CAD drawings of a dog-bone shape. Scale bars: 1 mm.

FIGS. 15B and 16B are scanning electron microscope images of 3D printed dog-bone shapes. Scale bars: 1 mm.

FIGS. 15C and 16C are scanning electron microscope images of PDMS molds. Scale bars: 1 mm.

FIG. 17A is a CAD drawing of a geodesic dome. Scale bar: 1 mm.

FIG. 17B is a scanning electron microscope image of a 3D printed geodesic dome. Scale bar: 1 mm.

FIG. 17C is a scanning electron microscope image of a PDMS mold of a geodesic dome. Scale bar: 1 mm.

FIGS. 18A and 18B are CAD drawings of micro-tug wells. FIGS. 18C and 18D are scanning electron microscope images of 3D printed micro-tug wells. FIGS. 18E and 18F are scanning electron microscope images of PDMS molds of micro-tug wells. Scale bars: 1 mm.

FIG. 19A is a CAD drawing of various dog-bone shapes having various lengths and shaft widths. Scale bars: 250 μm. FIGS. 19B, 19D, 19F, and 19H are scanning electron microscope images of dog-bone shapes having various lengths and shaft widths. FIGS. 19C, 19E, 19G, and 19I are scanning electron microscope images of dog-bone shapes having various lengths and shaft widths. Scale bars: 100 μm.

FIGS. 20A and 20B are CAD drawings of a pineapple house. FIGS. 20C and 20D are scanning electron microscope images of a 3D printed pineapple house. FIGS. 20E and 20F are scanning electron microscope images of a PDMS mold of a pineapple house. Scale bars: 1 mm.

FIG. 21 is a graph illustrating metabolic activity of C2C12 mouse cells when a 3D print, direct mold of PDMS, or a PDMS mold using a hydrogel intermediary mold were placed in the cells for 48 hours.

FIGS. 22A and 22B are brightfield images of C2C12 cells soaked in PDMS molds made directly from a 3D print for 48 hours. FIGS. 22C and 22D are brightfield images of C2C12 cells soaked in PDMS molds made using an intermediary hydrogel mold from a 3D print for 48 hours. FIG. 22E is a brightfield image of C2C12 cells after 48 hours. Scale Bars: 250 μm.

FIG. 23 is an image showing cardiomyocytes expressing striated sarcomeric α-actinin when micro-heart muscle is formed on a PDMS mold using a hydrogel intermediary mold.

FIG. 24A is a graph showing action potential of micro-heart muscle created using a PDMS mold from a hydrogel intermediary mold. FIG. 24B is a graph showing calcium transient waveforms of micro-heart muscle created using a PDMS mold from a hydrogel intermediary mold.

FIG. 25 is a scanning electron microscope image of an 8 post PDMS mold using the disclosed method.

FIG. 26 is a graph showing the success rate of the disclosed method using a laboratory detergent and not using a laboratory detergent.

FIGS. 27A-F are scanning electron microscope images of micro-tug PDMS molds formed using the disclosed method.

### DETAILED DESCRIPTION

The methods and resulting molds for use with cells will be understood, both as to its structure and operation, from the accompanying drawings, taken in conjunction with the accompanying description. It is noted that, for purposes of illustrative clarity, certain elements in various drawings may not be drawn to scale. Several variations of the device are presented herein. It should be understood that various components, parts, and features of the different variations may be combined together and/or interchanged with one another, all of which are within the scope of the present application, even though not all variations and particular variations are shown in the drawings. It should also be understood that the mixing and matching of features, elements, and/or functions between various variations is expressly contemplated herein so that one of ordinary skill in the art would appreciate from this disclosure that the features, elements, and/or functions of one variation may be incorporated into another variation as appropriate, unless described otherwise.

Several definitions that apply throughout this disclosure will now be presented. As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The terms “comprising” or “having” mean “including, but not necessarily limited to”; specifically indicate open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.” Specifically, the term “consisting essentially of” limits membership to the specified materials or steps and those that do not materially affect the essential characteristics of the claimed invention. The terms “a,” “an,” and “the” are understood to encompass the plural as well as the singular.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Disclosed herein is a method for replicating a 3D printed resin into a silicone rubber elastomer through a hydrogel intermediate. A hydrogel may be directly cast off a 3D printed resin and then a silicone rubber mold may be cast off the hydrogel, thus creating a copy of the 3D printed resin in the silicone rubber. The method may be extended to cast other materials that require free-radical mediated crosslinking, such as bisacrylamide hydrogels, or other types of elastomers, which would experience crosslinking inhibition due to the surface chemistry of the 3D printed resin and/or small molecules released by the 3D printed resin and the surface of the 3D printed resin, using the initial hydrogel as the intermediate.

The disclosed methods overcome previous limitations in PDMS molding off high-resolution stereolithographic (SLA) 3D prints. The disclosed double molding process allows for the replication of 3D printed features into PDMS, utilizing two classes of hydrogels as an intermediary. This allows for rapid design changes and overcomes limitations in soft lithography. Molding a hydrogel negative off the 3D print enables removal of the gel from the print with little/no plastic deformation of the gel to create a 1:1 PDMS replication of the original print. Further, the use of ductile hydrogels, such as agar and alginate hydrogels, is critical for replicating detailed features of a 3D print.

The method allows for more rapid dissemination of complex 3D devices, especially relevant to engineered tissues and the field of “organ-on-a-chip” products used for academic and pharmaceutical industry drug testing. In some aspects, the method may increase dissemination of 3D printed products, as the silicone rubber molds may be used to create shapes out of materials that cannot be 3D printed, including some plastics (e.g. polystyrene).

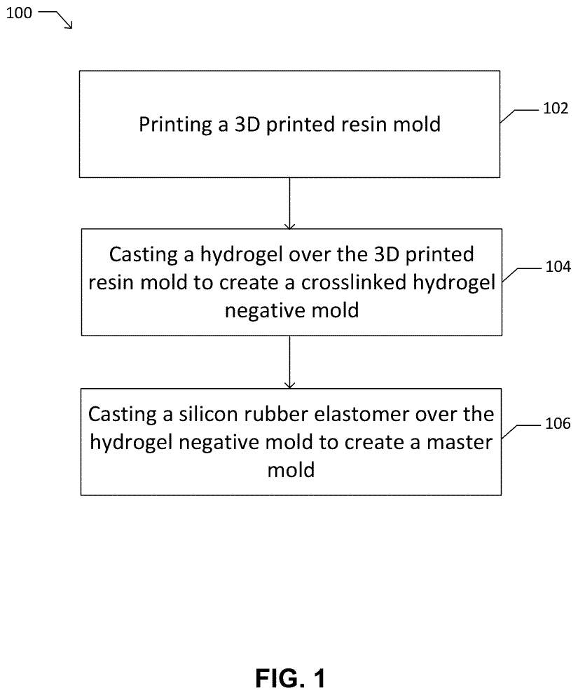

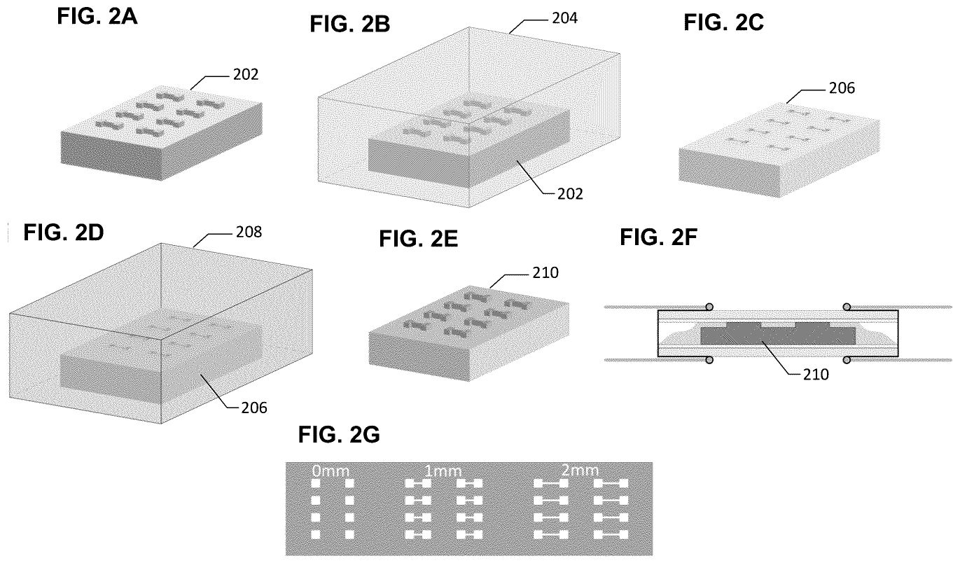

FIGS. 1-2G provide an overview of the disclosed method 100 of making a mold. At step 102, the method 100 may include printing a 3D printed resin mold. In some embodiments, poly(methacrylate) resins may be 3D printed using standard techniques known in the art. FIG. 2A further illustrates the 3D printed resin mold 202.

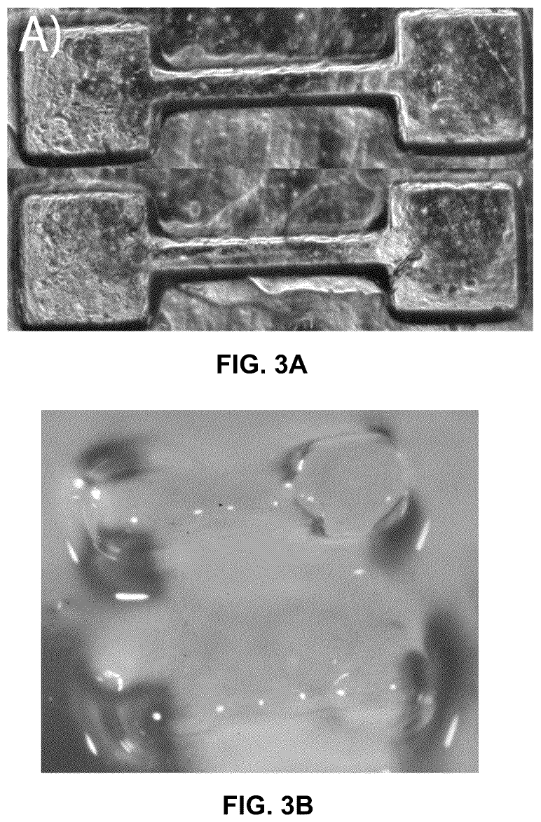

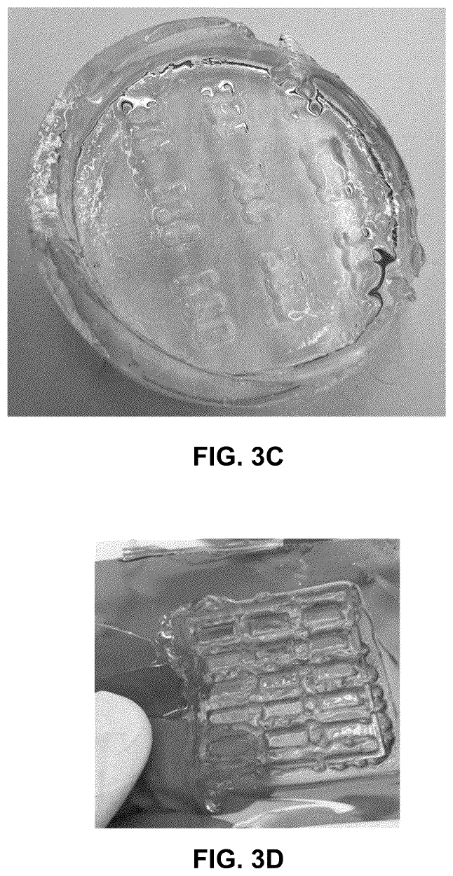

Casting silicone rubber elastomer on 3D printed resin inhibits the crosslinking of the silicone rubber elastomer. FIG. 3A is an example of a PDMS mold using standard photolithography techniques. While this method produces a sufficient mold, photolithography is limited in the height and detail of features that can be included in a 3D shape. FIGS. 3B-3D show examples of PDMS molds cast on 3D printed resin. As can be seen in the figures, the edges do not remain smooth or even appear to tear. Because of the difficulties in casting silicone rubber from 3D printed resin, using an intermediate between the resin and the silicone rubber may be beneficial in producing a silicone rubber mold that has the features of the 3D printed resin. Previously, epoxy has been attempted as an intermediary between a 3D print and PDMS. However, attempting to mold certain complicated shapes, such as hooks, with a stiff intermediary such as epoxy may cause the intermediary to break when it is removed off the print. In addition, the epoxy may still present potential limitations regarding surface chemistry compatibility. For example, an epoxy such as Ecoflex™ may have a surface chemistry that inhibits crosslinking. In fact, Ecoflex™ explicitly warns that curing of silicone rubber may be inhibited and may leave the rubber gummy or completely uncured.

Referring back to FIG. 1, at step 104, the method 100 may include casting a hydrogel over the 3D printed resin mold to create a crosslinked hydrogel negative mold. FIG. 2B further illustrates the hydrogel 204 cast over the 3D printed resin mold 202. FIG. 2C illustrates the crosslinked hydrogel negative mold 206. The use of a hydrogel as an intermediary may overcome the difficulties with either using an epoxy intermediary or directly molding from the 3D printed resin. For example, the use of hydrogels enables facile removal of the intermediary off the 3D printed resin mold due to the increased yield strain. In an embodiment, the hydrogel crosslinks through a mechanism that is not inhibited by small molecules released by the 3D printed resin and the 3D print surface. This mode of crosslinking may provide the optimal surface chemistry for crosslinking a silicone rubber like PDMS.

An additional advantage of using hydrogels is that the time of crosslinking, along with post-crosslinking hydrogel mechanical properties, can be engineered to optimize the double molding process. For example, the crosslink density and/or the toughness of the crosslinked hydrogel may be important features in allowing the silicone rubber to crosslink when in contact with the hydrogel. In various embodiments, the crosslinked hydrogel negative mold has a high crosslink density and/or a high toughness. In other words, the quality of the hydrogel for use as an intermediary mold increases as the crosslink density and the toughness increase. In another example, the replication fidelity (e.g., crosslinking success) may be impacted by the crosslink density, compressive toughness, and ultimate modulus. In some examples, the R² value of the predicted replication success based on the crosslink density may be about 0.60, about 0.65, or about 0.70. The R² value of predicted replication success based on the compressive toughness may be about 0.60, about 0.65, about 0.70, about 0.75, or about 0.80. The R² value of predicted replication success based on the ultimate modulus may be about 0.60, about 0.65, about 0.70, about 0.75, about 0.80, about 0.85, or about 0.90. In an example, the R² value of predicted replication success based on the crosslink density may be 0.70. The R² value of predicted replication success based on the compressive toughness may be 0.76. The R² value of predicted replication success based on the ultimate modulus may be 0.87.

In an embodiment, the crosslink density of the hydrogel may directly correlate to limiting, preventing, or reducing hydrogel water loss during PDMS crosslinking. In some examples, the toughness and ultimate modulus of the hydrogel may prevent or limit plastic deformation when the hydrogel is removed from the 3D-print.

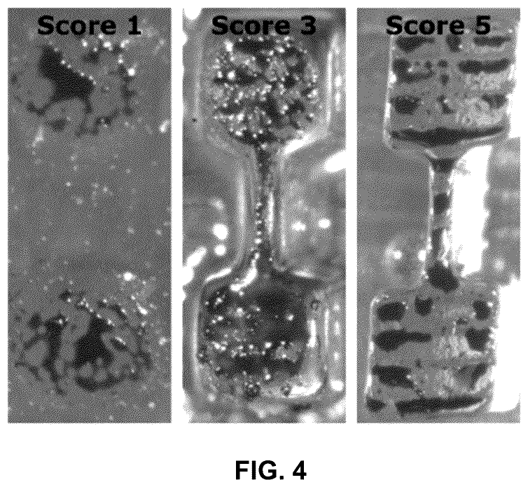

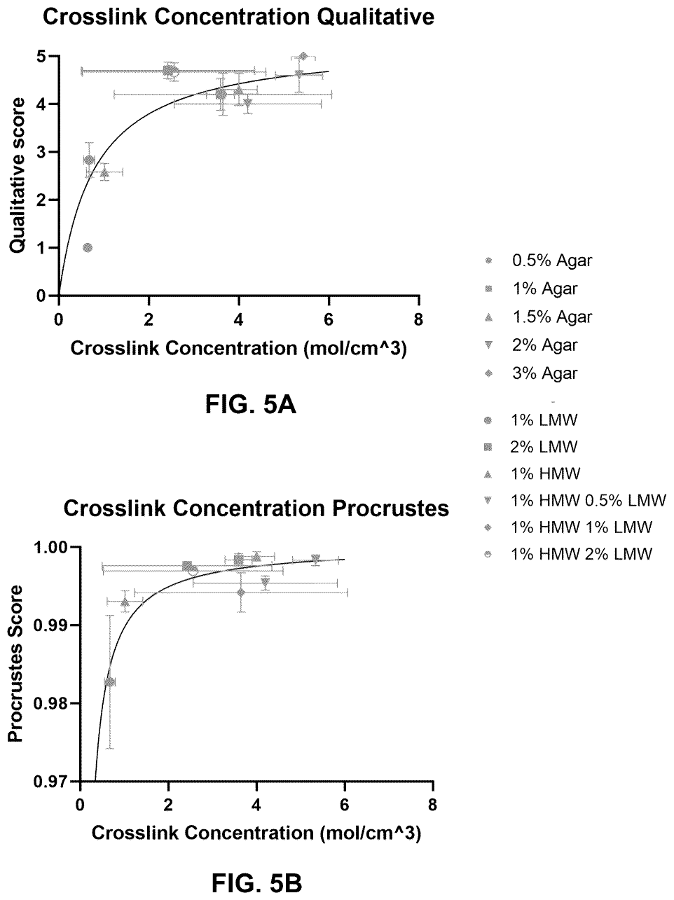

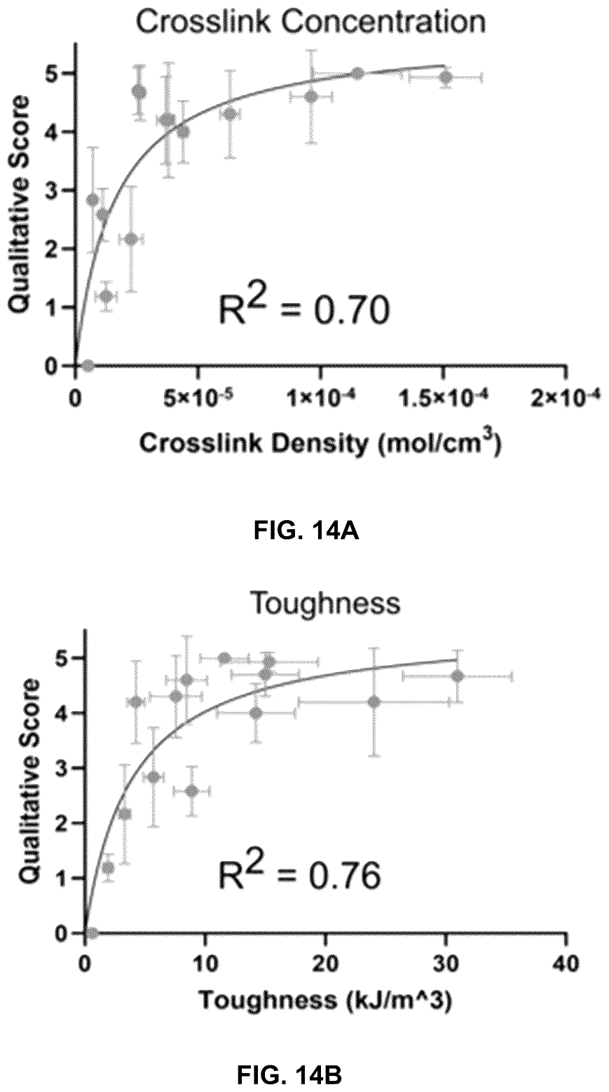

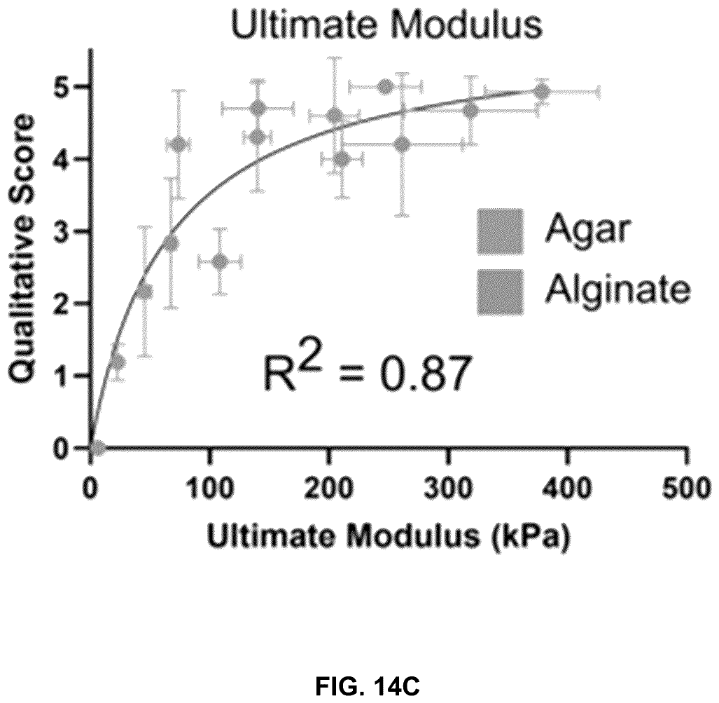

The master molds may be evaluated qualitatively and objectively using a 5 point observational scale or a Procrustes scale, respectively. FIG. 4 is an example of qualitative scoring. FIGS. 5A, 5B, and 14A are graphs of qualitative or Procrustes scores for PDMS molds using agar or alginate negative molds at various crosslink concentrations. The crosslink density of the hydrogel may be greater than about 2.5 mol/cm³ or greater than 4 mol/cm³. In some examples, the crosslink density of the hydrogel may be about 2.5 mol/cm³ to 3 mol/cm³, about 3 mol/cm³ to 3.5 mol/cm³, about 3.5 mol/cm³ to 4 mol/cm³, about 4 mol/cm³ to 4.5 mol/cm³, about 4.5 mol/cm³ to 5 mol/cm³, about 5 mol/cm³ to 5.5 mol/cm³, about 5.5 mol/cm³ to 6 mol/cm³, about 6 mol/cm³ to 6.5 mol/cm³, about 6.5 mol/cm³ to 7 mol/cm³, about 7 mol/cm³ to 7.5 mol/cm³, or about 7.5 mol/cm³ to 8 mol/cm³. The crosslink density of the hydrogel may lead to the master mold having a qualitative score of at least 3, at least 4, or at least 5. The crosslink density of the hydrogel may lead to the master mold having a Procrustes score of greater than 0.99, representing excellent fidelity of replication.

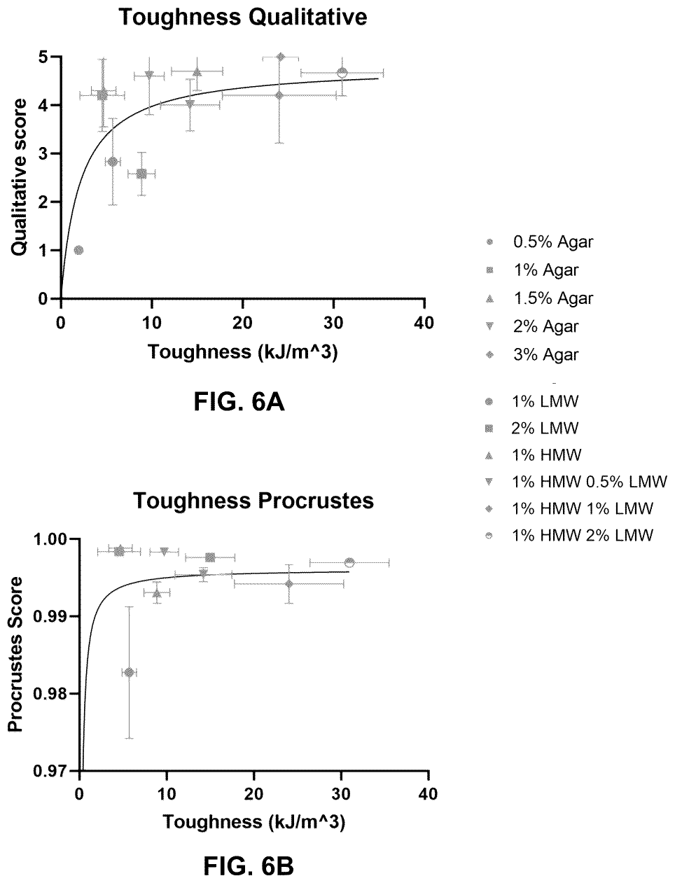

FIGS. 6A, 6B, and 14B are graphs of qualitative or Procrustes scores for PDMS molds using agar or alginate negative molds at various toughness's. The crosslinked hydrogel negative mold may have a toughness of greater than 8 kJ/m³. In some examples, the toughness of the hydrogel may be about 8 kJ/m³ to 10 kJ/m³, about 10 kJ/m³ to 15 kJ/m³, about 15 kJ/m³ to 20 kJ/m³, about 20 kJ/m³ to 25 kJ/m³, about 25 kJ/m³ to 30 kJ/m³, about 30 kJ/m³ to 35 kJ/m³, or about 35 kJ/m³ to 40 kJ/m³. The toughness of the hydrogel may lead to the master mold having a qualitative score of at least 3, at least 4, or at least 5. The toughness of the hydrogel may lead to the master mold having a Procrustes score of greater than 0.99, representing excellent fidelity of replication.

FIG. 14C is a graph of qualitative scores for PDMS molds using agar or alginate negative molds at various ultimate modulus's. The crosslinked hydrogel negative mold may have an ultimate modulus of greater than 20 kPa. In some examples, the ultimate modulus of the hydrogel may be about 20 kPa to about 40 kPa, about 40 kPa to about 60 kPa, about 60 kPa to about 80 kPa, about 80 kPa to about 100 kPa, about 100 kPa to about 120 kPa, about 120 kPa to about 140 kPa, about 140 kPa to about 160 kPa, about 160 kPa to about 180 kPa, about 180 kPa to about 200 kPa, about 200 kPa to about 220 kPa, about 220 kPa to about 240 kPa, about 240 kPa to about 260 kPa, about 260 kPa to about 280 kPa, about 280 kPa to about 300 kPa, about 300 kPa to about 320 kPa, about 320 kPa to about 340 kPa, about 340 kPa to about 360 kPa, about 360 kPa to about 380 kPa, about 380 kPa to about 400 kPa, about 400 kPa to about 420 kPa, about 420 kPa to about 440 kPa, about 440 kPa to about 460 kPa, about 460 kPa to about 480 kPa, or about 480 kPa to about 500 kPa. The ultimate modulus of the hydrogel may lead to the master mold having a qualitative score of at least 3, at least 4, or at least 5.

In some embodiments, the hydrogel may be agar, alginate, hyaluron or gelatin. In some examples, the hydrogel includes about 0.5% w/v to 1% w/v, about 1 w/v to 1.5% w/v, about 1.5% w/v to 2% w/v, about 2% w/v to 2.5% w/v, or about 2.5% w/v to 3% w/v agar. In at least one example, the hydrogel includes 1.5% w/v agar.

In some embodiments, the agar hydrogel may be formed by dissolving agar in water and heating the mixture to a boil. Then the hydrogel may be allowed to cool to below the glass transition phase temperature of the 3D print material before casting the hydrogel on to the 3D print. In an example, the agar hydrogel may be formed by dissolving agar in tap water, microwaving the mixture for 2:00 minutes, then allowing it to cool to below 60 degrees C. before pouring the hydrogel onto the 3D-print. The hydrogel on the 3D-print may then be chilled in a refrigerator having a temperature of about 4 degrees C. for 20 to 30 minutes.

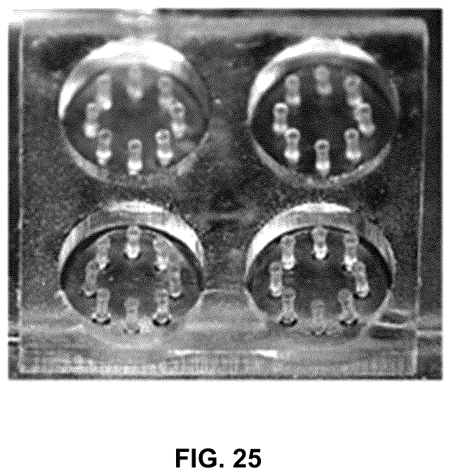

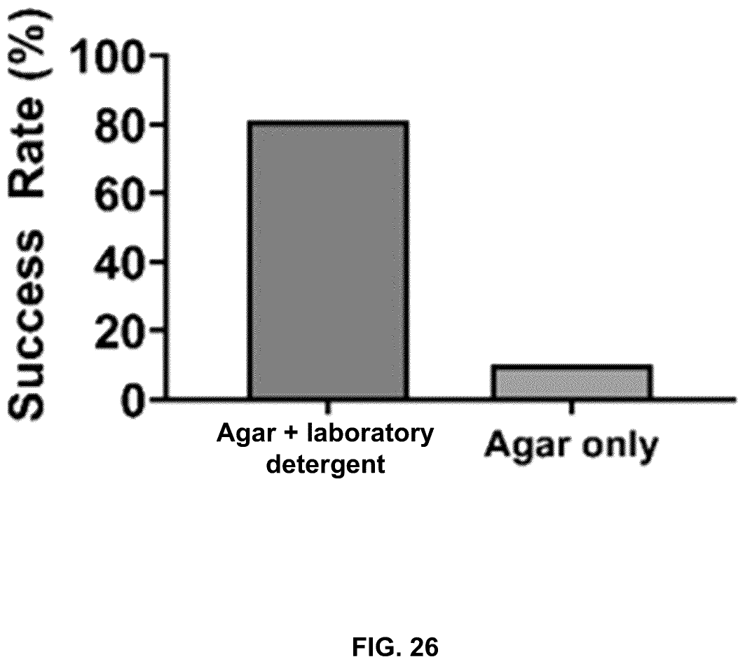

As illustrated in FIG. 25, a laboratory detergent (e.g., Triton X-100) may be added to the agar hydrogel. In some examples the detergent may be present in the hydrogel at concentration of about 0.05%, about 0.1%, about 0.15%, about 0.20%, about 0.25%, about 0.30%, about 0.35%, about 0.40%, about 0.45%, or about 0.50%. In another example, the detergent may be present in the hydrogel at a concentration of about 0.05% to about 0.1%, about 0.1% to about 0.15%, about 0.15% to about 0.20%, about 0.20% to about 0.25%, about 0.25% to about 0.30%, about 0.30% to about 0.35%, about 0.35% to about 0.40%, about 0.40% to about 0.45%, or about 0.45% to about 0.50%. The laboratory detergent may reduce the surface tension between the hydrophilic gels and the hydrophobic elastomers (e.g., PDMS) during crosslinking to achieve a high-aspect ratio of the 3D-print mold in the elastomer mold (e.g., PDMS mold). As illustrated in FIG. 26, the laboratory detergent may improve the success rate of the disclosed method by about 70%. In other examples, the use of a laboratory detergent mixed with the hydrogel may provide an 80% success rate of the disclosed method. In further examples, mixing a laboratory detergent with the hydrogel before casting or during casting may result in an increased successful PDMS mold rate compared to a hydrogel without a laboratory detergent.

In additional examples, the hydrogel may include 1% w/v to 2% w/v high molecular weight (HMW) alginate and/or 0% w/v to 2% w/v low molecular weight (LMW) alginate. In at least one example, the hydrogel may include 1% high molecular weight alginate and 2% low molecular weight alginate. In some examples, forming the hydrogel may comprise dissolving a 3% w/v (HMW) alginate or a 5% w/v (LMW) alginate in 0.1 M 2-(N-morpholino) ethane sulfonic acid (MES) buffer with a pH between 6 to 7. In some examples, the alginate hydrogel may comprise 1% LMW, 2% LMW, 1% HMW, 1 HMW+0.5% LMW, 1% HMW+1% LMW, and 1% HMW+2% LMW. In other examples, the 0.1 M 2-(N-morpholino) ethane sulfonic acid (MES) buffer may be substituted with other buffers having similar chemical properties.

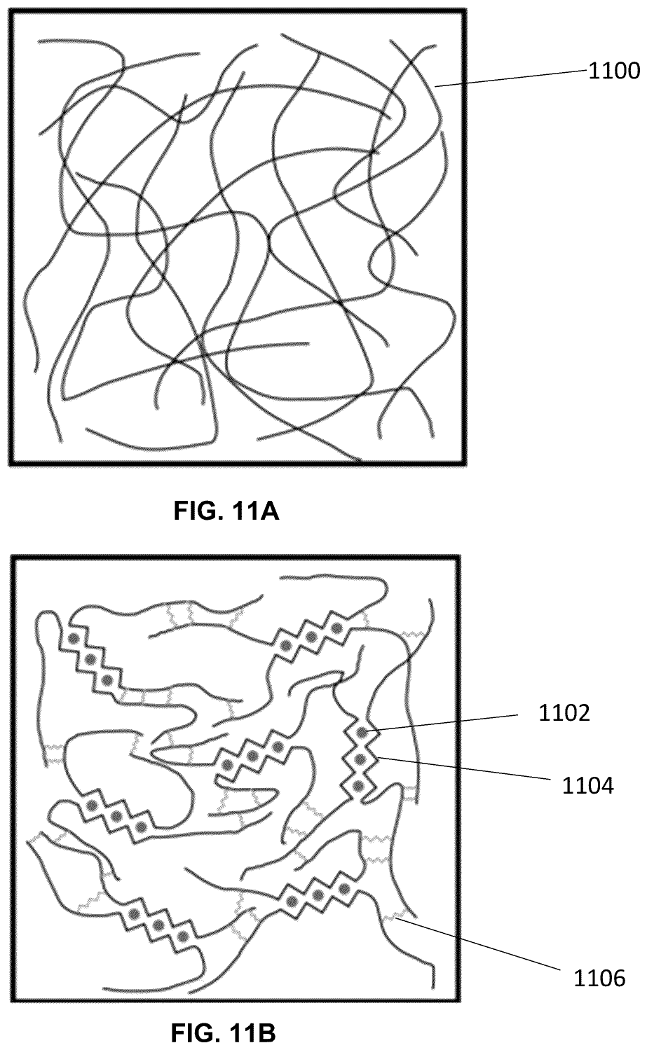

In some embodiments, the hydrogel crosslinking mechanisms may depend on the type of hydrogel used. Agar may be crosslinked by physical entanglement and alginate may be crosslinked by covalent and ionic bonds. Both the physical entanglement crosslinking mechanism and the covalent and ionic bonds crosslinking mechanism may be unaffected by unreacted monomers and photo-inhibitors that may be present in 3D printed materials. As illustrated in FIG. 11A, for an agar hydrogel a physical entanglement crosslinking mechanism may be used to form the hydrogel. The physical entanglements 1100 are illustrated in FIG. 11A.

As illustrated in FIG. 11B, for an alginate hydrogel a covalent and ionic bonds crosslinking mechanism may be used. As illustrated in FIG. 11B, calcium²⁺ ions 1102 may be used to form the ionic crosslinks 1104. In some examples, the covalent bonds may be carbodiimide and the ionic bonds may be calcium. In an example, covalent crosslinks 1106 may be formed using adipic-acid-dihydrazide/1-ethyl-3-(3-dimethylaminopropyl)-carbodiimide). In some examples, to enable flow-forming ionic bonds, a salt (e.g., calcium chloride) may be bound to ethylenediaminetetraacetic acid and dissolved in 0.1M MES at a pH of approximately 7.2. In other examples, covalent and ionic bonds may be formed using other chemicals known in the art to produce covalent and ionic bonds for crosslinking.

In some embodiments, the fully crosslinked alginate hydrogel negative may be soaked in a buffer with a pH between 7 to 10. The crosslinked alginate hydrogel may be soaked for about 9 hours to about 14 hours. In some examples, after soaking, the fully crosslinked alginate hydrogel may be heated to further promote PDMS crosslinking. In some examples, fully crosslinked alginate hydrogel negatives of the 3D-prints may be soaked in about 0.1 M MES containing 12.5 mM CaCl₂ with a pH between 8-9 for 12 hours, then removed and placed in a 37 degree C. oven for about 1 hour to further promote PDMS crosslinking.

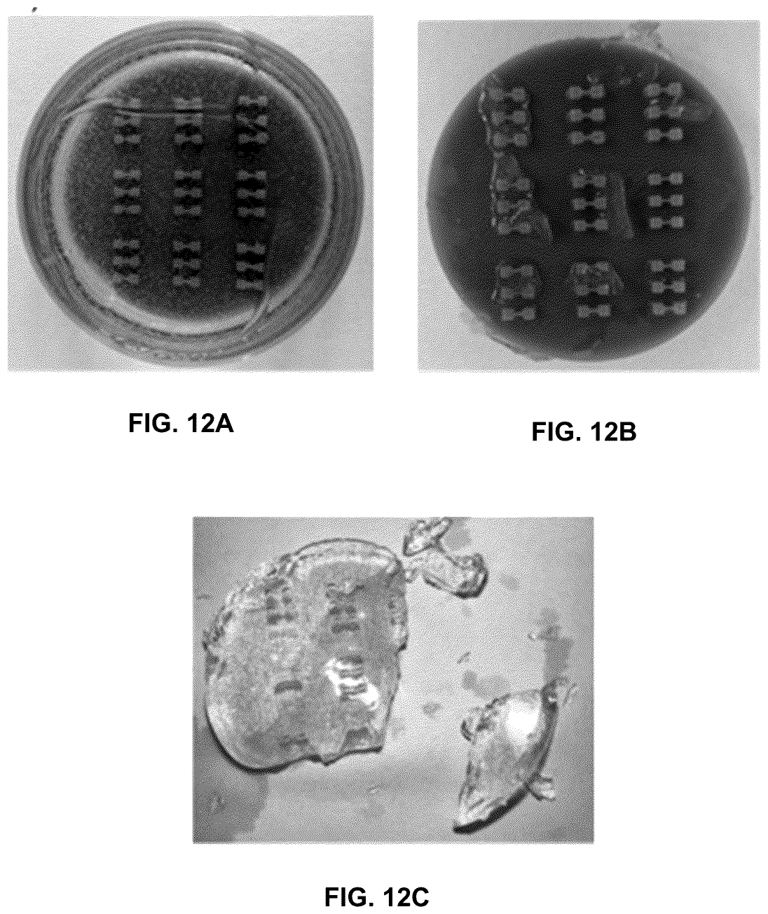

FIGS. 12A-C show images of brittle alginate hydrogels that only use covalent bond crosslinking. The resulting hydrogel is brittle and may be prone to cracking producing a low fidelity PDMS mold. By using a crosslinking mechanism of both covenant and ionic bonds the resulting PDMS mold may be of a high fidelity.

Referring back to FIG. 1, at step 106, the method 100 may include casting a silicone rubber elastomer over the hydrogel negative mold to create a master mold. FIG. 2D illustrates the silicone rubber elastomer 208 cast over the hydrogel negative mold 206. FIG. 2E illustrates the master mold 210 made of the silicone rubber elastomer 208. In an embodiment, the silicone rubber elastomer is poly(dimethyl siloxane) (PDMS). In other embodiments, the master mold may be made from another material that would normally not crosslink efficiently on the surface of the SLA 3D print, such as a polyacrylamide gel crosslinked using bis-acrylamide. The master mold may be a 1:1 replication of the 3D printed resin mold. In some examples, the resin may be a clear resin, a gray resin, white resin, or other resin used in 3D printing. In some examples, the resin may be acrylonitrile butadiene styrene (ABS-like resin), polylactic acid resin (PLA), thermoplastic polymer resin (e.g., PETG), nylon resin, polycarbonate resin, and/or other resins known in the art. In another examples, the resin may be standard resin, clear resin, tough resin, flexible resin, water-washable resin, and/or other resins known in the art. In some examples, the master mold replicates up to 15 μm z-axis resolution of the 3D printed resin mold. In other examples, the master mold replicates features as small as 10 μm from the 3D printed resin mold. In some embodiments, the master mold may have a height greater than 2 mm or have one or more features taller than 2 mm.

In some embodiments, step 106 may include mixing the silicone rubber elastomer, pouring it into the hydrogel negative, degassing the silicon rubber elastomer, and curing the silicon rubber elastomer at a temperature for a period of time. In some examples, the silicon rubber elastomer may be cured at 37 degrees C. for at least 8 hours to form the master mold 210. In other examples, the silicon rubber elastomer may be cured at a temperature of about 25 degrees C. to about 30 degrees C., about 30 degrees C. to about 35 degrees C., about 35 degrees C. to about 40 degrees C., or about 40 degrees C. to about 45 degrees C. In another example, the silicon rubber elastomer may be cured for about 5 hours to about 6 hours, about 6 hours to about 7 hours, about 7 hours to about 8 hours, about 8 hours to about 9 hours, about 9 hours to about 10 hours, about 10 hours to about 11 hours, or about 11 hours to about 12 hours. During the curing process, an oven may be used containing at least about 3 liters of water within a large jug to maintain humidity to prevent excess hydrogel water loss during PDMS crosslinking. In some examples, the oven may contain about 3 liters to about 4 liters of water, about 4 liters to about 5 liters of water, or about 5 liters of water to about 6 liters of water.

In some embodiments, the master mold may be capable of replicating features of 3D prints on the scale of 10 μm. The master mold may be used in a wide range of sizes and geometries without deformation to the original print, enabling successive replication off a single 3D print.

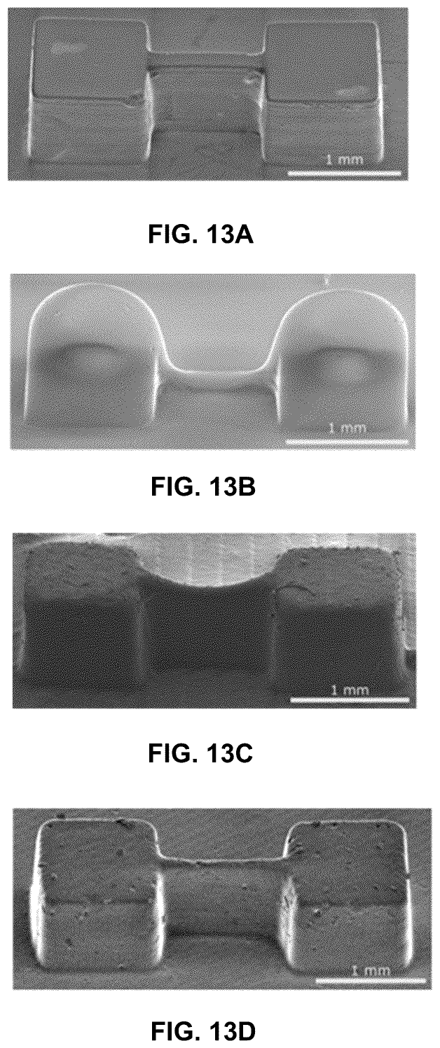

FIGS. 13B-D illustrate representative PDMS replications of a 3D printed dog-bone shape using hydrogels as an intermediary mold. FIGS. 13A-D were obtained using scanning electron micrographs. FIG. 13A shows the original 3D printed dog-bone shape. FIG. 13B shows the representative PDMS replication having a qualitative score of 3. FIG. 13C shows the representative PDMS replication having a qualitative score of 4. FIG. 13D shows the representative PDMS replication having a qualitative score of 5. As illustrated in FIGS. 13B-D, the PDMS replications of the dog-bone shape may maintain the detail and features of the 3D print illustrated in FIG. 13A.

Molds cast directly from a 3D printed resin could potentially have toxins from the resin leach into the mold, making the mold unsuitable for use with cells. The intermediate step of using the hydrogel may prevent the leaching of any toxins from the 3D printed resin mold. In an embodiment, the master mold does not contain any leachate from the 3D printed resin mold. Thus, the master mold may be biocompatible and does not contain any toxins that would inhibit cell growth, even if the material used for the 3D printing is toxic or produces toxic leachate.

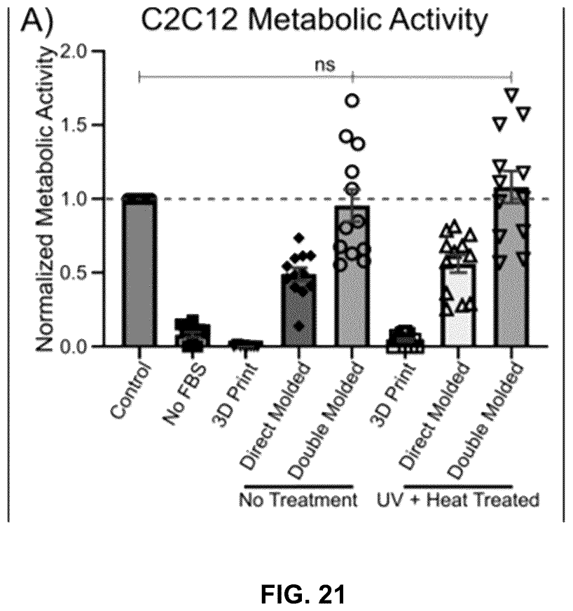

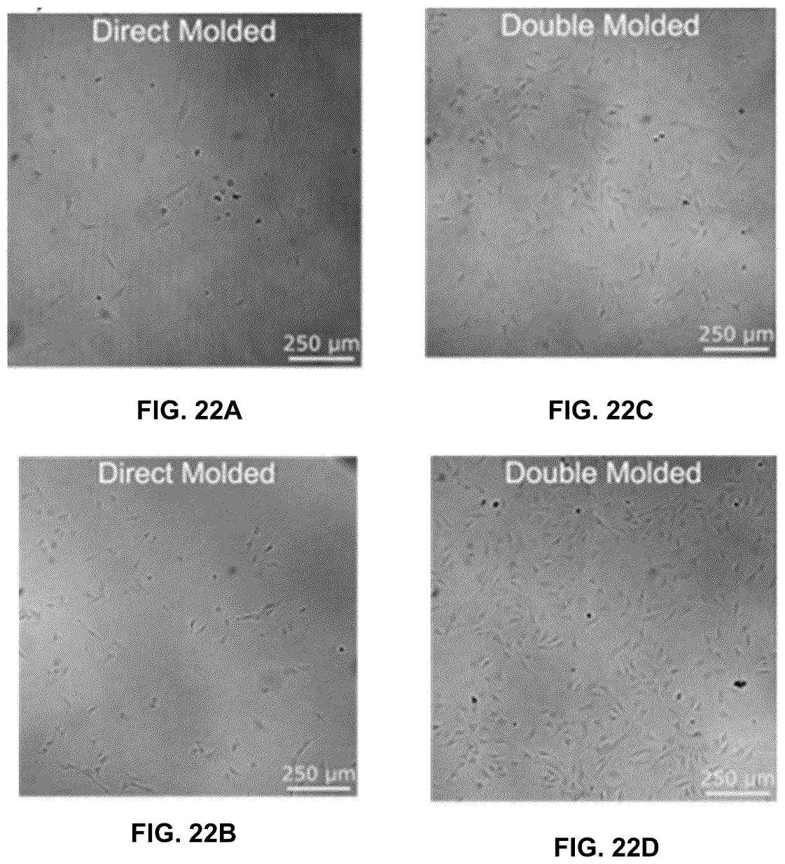

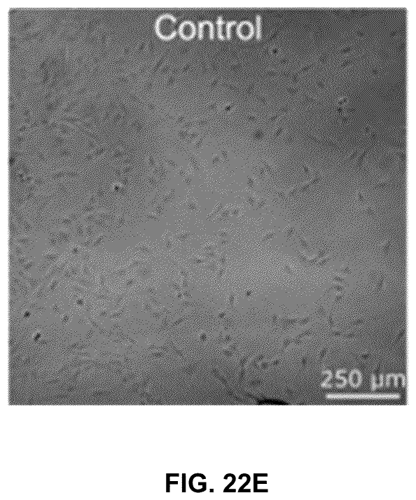

FIGS. 21 and 22A-D illustrate results of metabolic activity when soaking C2C12 cells in a 3D print mold, a direct mold of a silicon rubber elastomer from a 3D print, the silicon rubber elastomer mold formed using the method disclosed herein, and a control group. The cells were soaked for 48 hours.

FIG. 21 illustrates a graph showing the difference in metabolic activity of C2C12 cells after being soaked with a 3D print directly, a direct molded silicon rubber elastomer (e.g., casting the silicon rubber elastomer directly on to the 3D print), and the double molded disclosed method (e.g., using a hydrogel intermediary). As illustrated in FIG. 21, the comparative toxicity levels of the 3D print, a direct molded PDMS mold and the double molded master mold using the disclosed method produce very different toxicity levels. The master mold created using the disclosed method may display metabolic activity levels similar to the control group, indicating little to no toxicity. The level of metabolic activity for the master mold creating using the disclosed method indicates little to no leaching of the toxins from the resin of the 3D print. FIG. 21 also shows that the disclosed method produces a master mold that does not need UV and heat treatments to produce a biocompatible master mold that does not contain any toxins that inhibit cell growth.

As illustrated in FIGS. 22A and 22B, the brightfield images of a direct molding of a silicon rubber elastomer off of a 3D print exhibit limited metabolic activity of cells after 48 hours. In FIGS. 22C and 22D, the brightfield images of a silicon rubber elastomer formed using the disclosed method displays similar metabolic activity to the brightfield image of a control group of cells exhibited in FIG. 22E, indicating the master mold formed using the disclosed method contains little to no toxicity.

In other embodiments, the method 100 may include casting an additional object from the master mold. For example, the master mold may be used to create objects/shapes out of materials that cannot be 3D printed, including but not limited to plastics such as polystyrene.

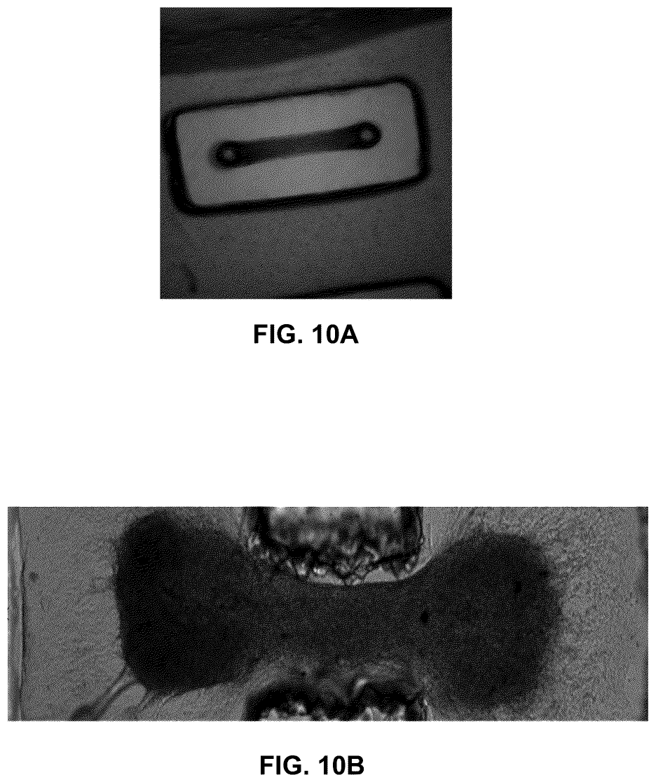

The master mold may then be used to hold cells for tissue engineering, creating a microfluidic “organ-on-a-chip”, or other research purposes. FIG. 2F shows an example of the master mold being used to crosslink PDMS devices with dog-bone-shaped “through-holes”. FIG. 2G shows an example of such a stencil with through-holes of various geometries and sizes. FIGS. 10A and 10B are example images of engineered tissue created using the PDMS mold made using the disclosed method.

The master mold may be used to create engineered tissues. In one example, the engineered tissue may be a micro-heart muscle (e.g., micro-heart cells). In some examples, the engineered tissue may comprise cardiomyocytes and stromal cells, such as fibroblasts and/or mesenchymal stem cells. In other examples, the engineered tissues may be any organ tissue or other biological tissue. The engineered tissue may model in vivo organ systems. The engineered tissues may have geometries and functions similar to or the same as in vivo organ systems. These engineered tissues may be used in a variety of research applications to study effects on organ systems. In some embodiments, the engineered tissue may be formed by using the master mold of the disclosed method and seeding the master mold with cells of the desired tissue. In some examples, the cells may be cardiomyocytes.

In some embodiments, the master mold may be placed into a tissue culture device (e.g., tissue culture well plate). The PDMS master mold may be dipped in methanol and then placed into the wells of the tissue culture well plate. The well plate may then be placed in an oven to reversibly bond the PDMS mold to the well surface. The wells and PDMS molds may be sterilized by soaking in 70% ethanol overnight, then coated with fibronectin to enable cell attachment to the substrate. In some examples, desired cells may then be seeded and incubated at about 37 degrees C. for about 60 minutes before adding media.

### EXAMPLES

### Example 1

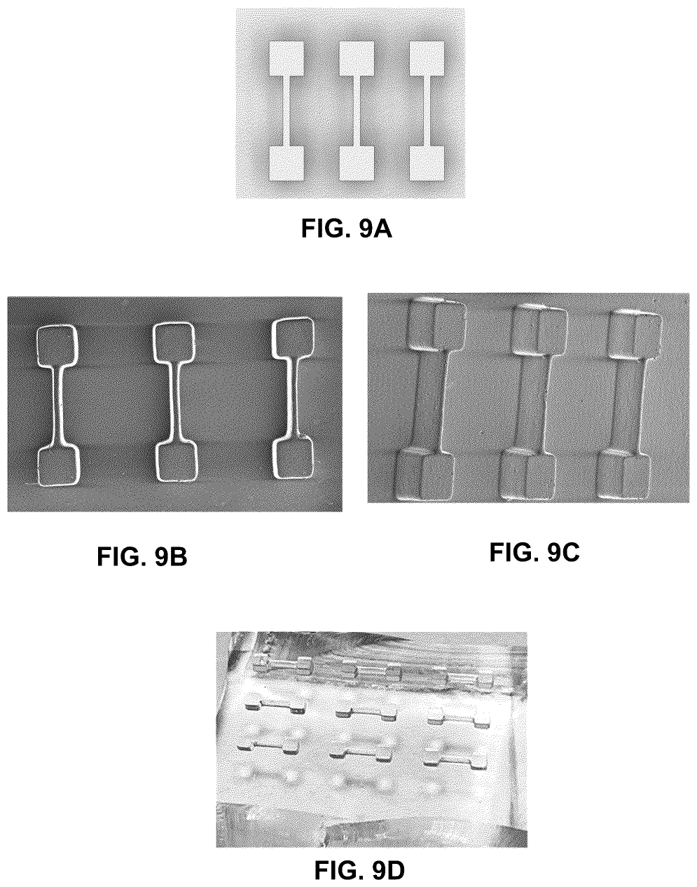

To quantify how polymer concentration and the resulting gel mechanical properties correlate with replication fidelity, agar and alginate hydrogels were created at varying concentrations and were used to replicate dog-bone features into PDMS. Specifically, agar was used at 0.5-3.0% w/v and the HMW alginate was used at 1-2% w/v combined with 0-2% w/v of the LMW alginate. First, the PDMS replications were qualitatively quantified for their replication fidelity based on observer analysis, where the replicated PDMS dog-bones (e.g. FIGS. 9A-9D and 15A-16C) were given a score on a 1-5 scale, with 5 being a visually perfect feature replication and 1 represents a complete failure to replicate. FIGS. 13B-D illustrate example qualitative scores. FIG. 13B was given a qualitative score of 3. FIG. 13C was given a qualitative score of 4. FIG. 13D was given a qualitative score of 5. Example images of the replications showing a variety of representative replications and the qualitative score assigned are shown in FIG. 4. The replication capability of the hydrogels was then also objectively quantified in a user-independent fashion. The 2D dog-bone shape of the replication was outlined, which were then compared to the original 3D print using the Matlab Procrustes function.

It was found that increasing concentrations of agar led to increased replication fidelity, both qualitatively and quantitatively (FIGS. 5A-6B and 14A). However, increasing the agar concentration further (above 5%) inhibited the replication as the kinetics of crosslinking were too rapid for the pre-gel polymers to conform to the print (not shown). A similar result was also found in alginate double-network hydrogels, however the HMW alginate networks needed a minimum amount of LMW chains to achieve reasonable levels of replication fidelity, which are likely necessary to properly enable ionic bonding. In order to determine the characteristics that regulate the hydrogels' ability to accurately replicate 3D features, a wide range of materials' mechanical properties were investigated. Crosslink density had a strong correlation to replication fidelity and toughness provided a threshold-effect to replication fidelity (FIGS. 5A-6B and 14A-B). Further, ultimate modulus had a strong effect on replication fidelity (FIG. 14C). It is hypothesized that the crosslink density toughness, and/or ultimate modulus have an effect on water retention in the gel to maintain its shape, which may then lead to higher fidelity in PDMS molds. Moreover, the kinetics of crosslinking should be slow enough that the pre-gel polymer can conform to the micro-scale features of the 3D print.

Although agar was able to achieve higher replication scores both qualitatively and quantitatively with increasing concentration, it was found that the increased stiffness in agar above 1.5% w/v made it practically difficult to remove the gel from the 3D print. Increasing agar concentrations also increased viscosity and gelation speed, often leading to air bubble formation and decreased replication efficiency. For simple 2D extruded shapes up to ˜2 mm in height, 1.5% w/v agar provided adequate replication. For more complex 3D features or those with larger height, it was found that the use of the tougher 1% HMW/2% LMW alginate may be better suited for enabling removal of the hydrogel from the print. For example, when replicating rings that had a wall thickness and height of several mm, the agar tore when attempting to remove the relatively thin gel due to the high amount of surface contact with the print.

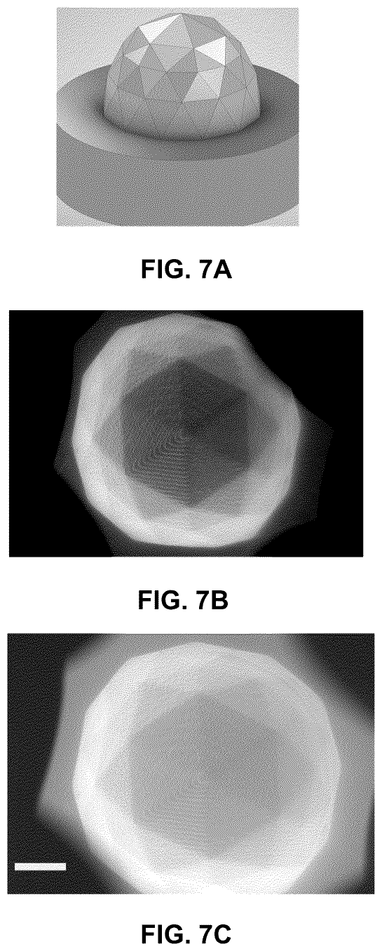

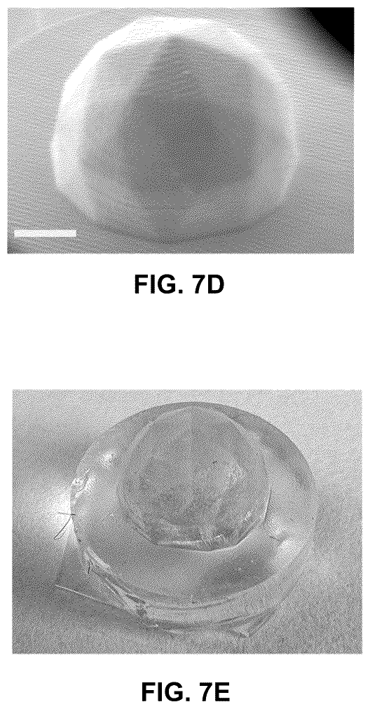

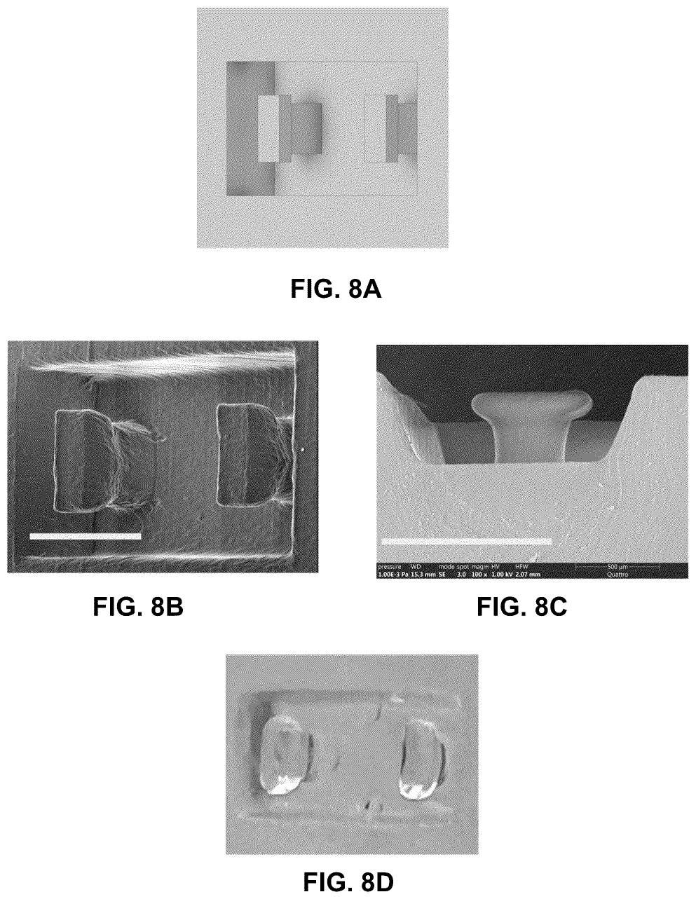

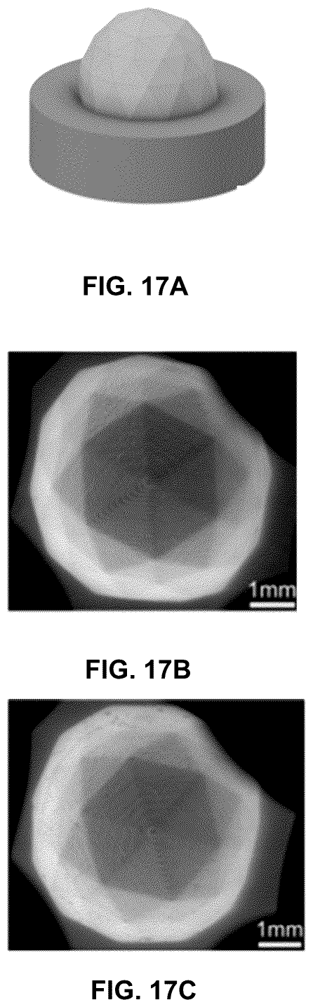

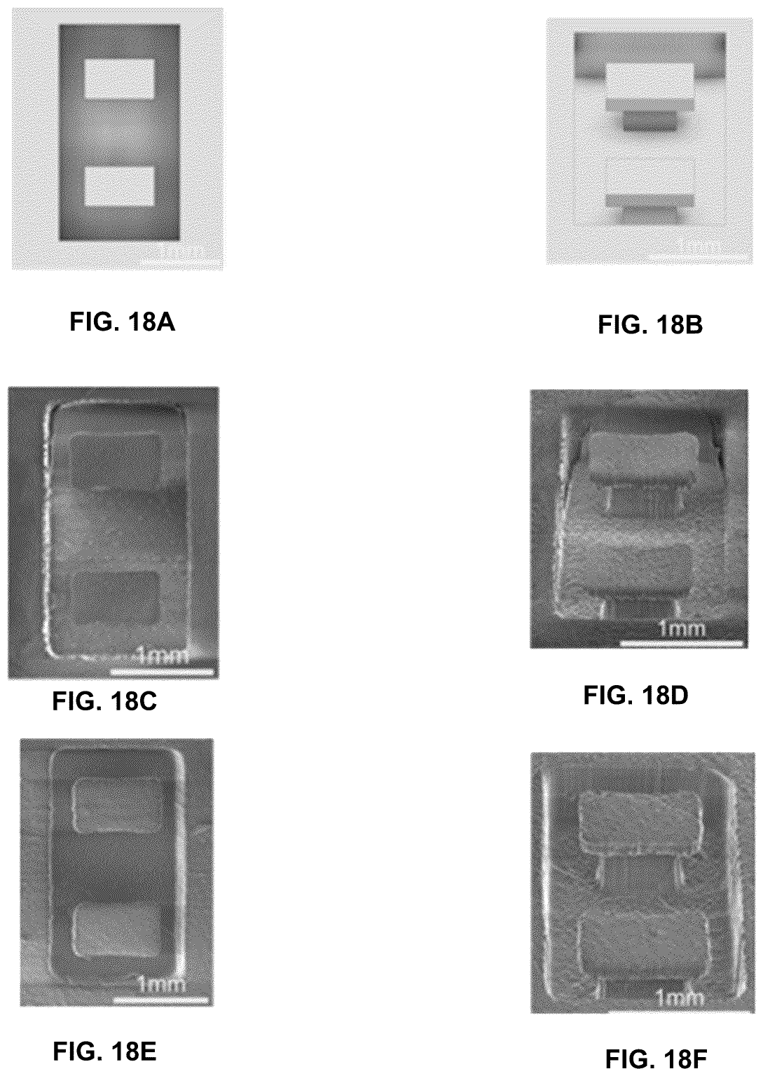

Several features that are difficult or impossible to create using standard soft lithography were 3D printed, including a geodesic dome, micro-tugs, and dog-bone structures as seen in FIGS. 7A-7E, 8A-8D, 9A-9D, 15A-C, 16A-16C, 17A-C, and 18A-F. Scanning electron microscope imaging of these features highlights the resolution at which the method is able to replicate 3D printed features. In addition to the 1:1 reproduction, the ˜15 μm z-axis resolution of the 3D printer used is reproduced in the final PDMS replicate as shown in the geodesic dome, dog-bone shapes, and micro-tugs (FIGS. 7C, 8C, 9C, 15C, 16C, 17C, 18E, and 18F). The micro-tugs could not be formed by direct casting off a 3D print because the elastomer would break off due to the large lateral dimension of the “caps” on top of the pillars.

### Example 2

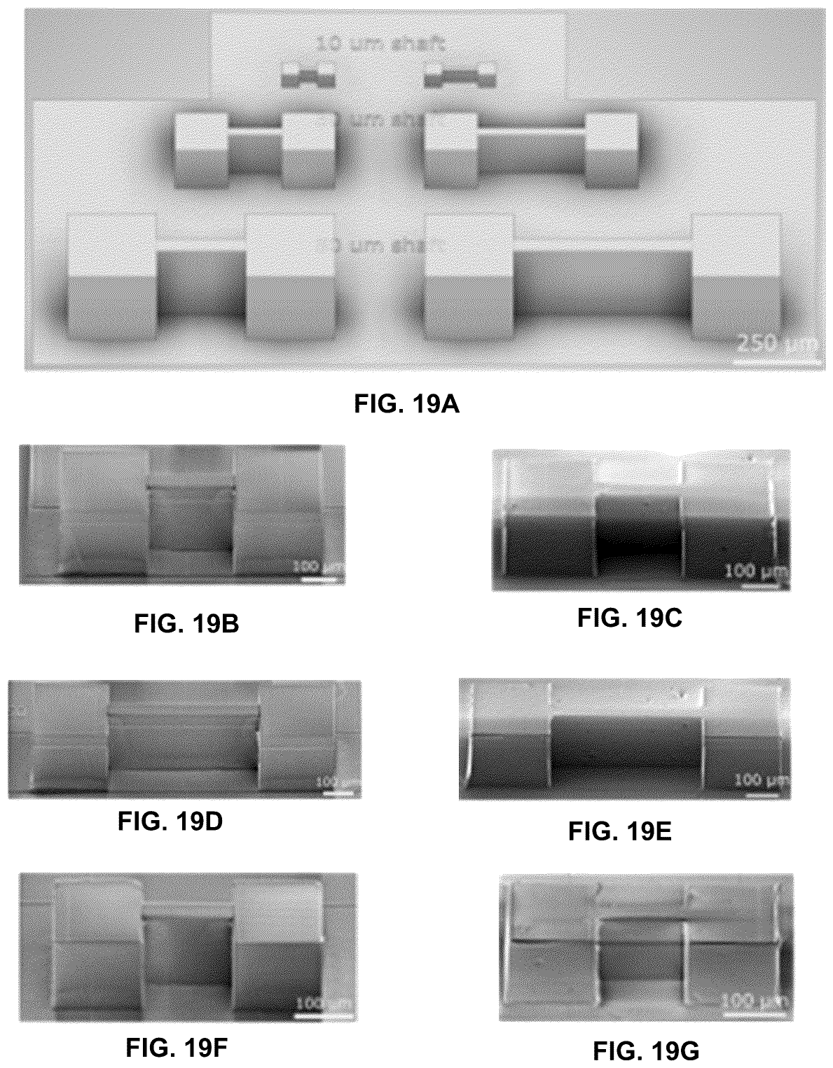

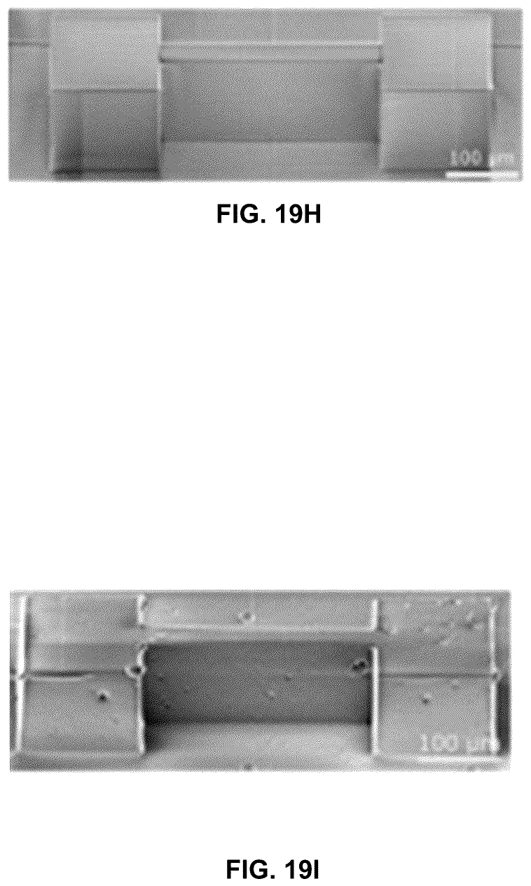

To further highlight the capabilities of the disclosed method, dog-bone molds were printed using a Nanoscribe printer. The Nanoscribe printer uses 2-photon generated light to achieve printing resolutions more than an order of magnitude smaller than the finest features allowed by standard single-photon stereolithographic printers like the printer used for FIGS. 7A-7E, 8A-8D, 9A-9D, and 15A-18F. The dog-bone molds maintained the same dimension ratios as the previously used designs, but were 4 to 20 times smaller, as illustrated in FIGS. 19A-I. FIG. 19A illustrates CAD drawings of 3D prints for FIGS. 19B-I. FIG. 19B illustrates a dog-bone 3D print having a length of 750 μm and a shaft width of 50 μm. FIG. 19D illustrates a dog-bone 3D print having a length of 1000 μm and a shaft width of 50 μm. FIG. 19F illustrates a dog-bone mold having a length of 450 μm and a shaft width of 30 μm. FIG. 19H illustrates a dog-bone mold having a length of 600 μm and a shaft width of 30 μm.

A 2.5% w/v agar hydrogel was used as an intermediary mold for the master PDMS mold of FIGS. 19C, 19E, 19G, and 19I. FIG. 19C is the PDMS mold from the 3D-print of FIG. 19B using the disclosed method. FIG. 19E is the PDMS mold from the 3D-print of FIG. 190 using the disclosed method. FIG. 19G is the PDMS mold from the 3D-print of FIG. 19F using the disclosed method. FIG. 19I is the PDMS mold from the 3D-print of FIG. 19H using the disclosed method. As illustrated in FIGS. 19C, 19E, 19G, and 19I, PDMS master mold replications of the 3D prints using the disclosed method were achieved with minimal deformation, although the corners on one side of the PDMS dog-bone has small, sharp protrusions not present in the original 3D-print. These are likely due to stress concentrations at the sharp corners that cause microtears in the agar when it is removed from the print. However, because these microtears are much smaller than the scale of multicellular tissues, deformation on this small scale would be unlikely to affect the usefulness of the PDMS replica for engineered tissue formation.

### Example 3

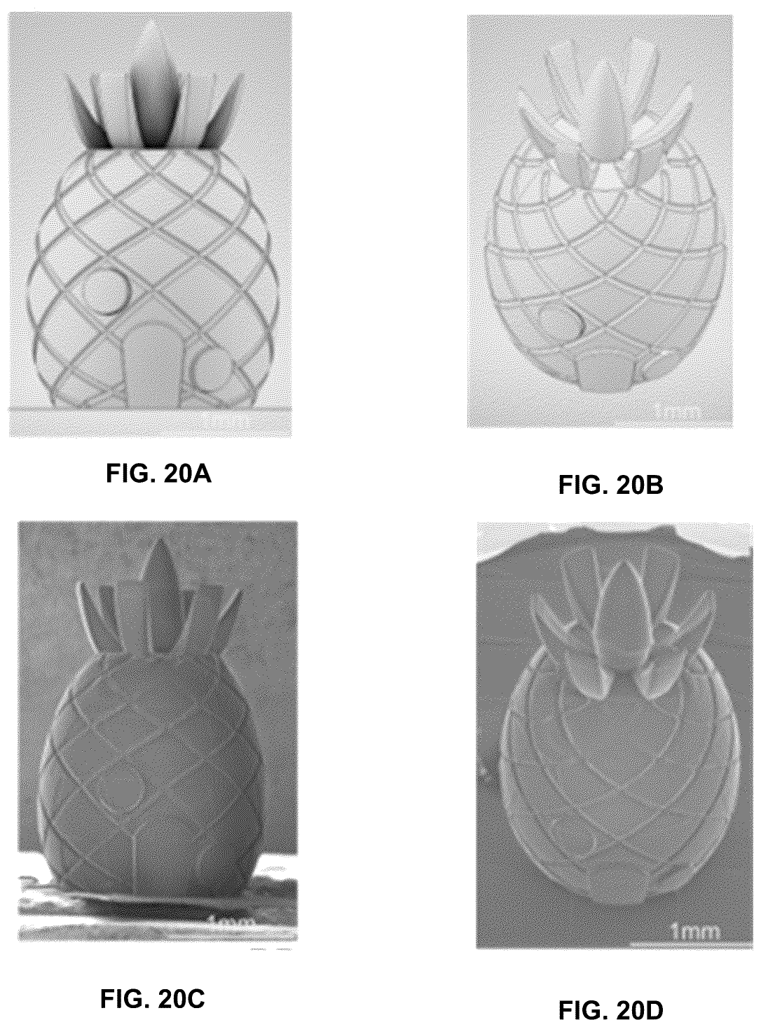

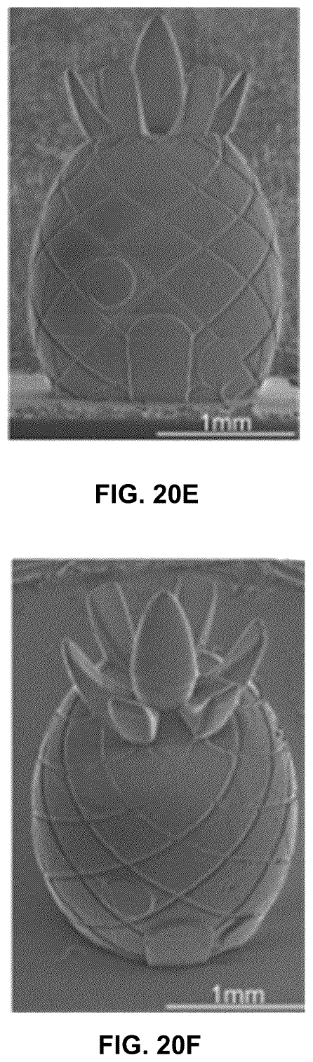

To feature the capabilities of the hydrogels and show the ability of hydrogels to replicate a wide variety of shapes at different size scales, the Nanoscribe printer was used to 3D-print a pineapple house with many fine details. A 2.5% w/v agar hydrogel was used as the intermediary mold. FIGS. 20A-B illustrate CAD drawings of the pineapple house. FIGS. 20C-D illustrate 3D prints of the pineapple house. FIGS. 20E-F illustrate the PDMS mold made using the hydrogel from the 3D-print of FIGS. 20B-C. As illustrated in FIG. 20C-D, using the hydrogel as an intermediary mold allowed production of the PDMS mold while maintaining detail and fidelity. The groves in the pineapple house were 50 μm in width and created the distinct diamond pattern, which can be completely seen in the PDMS mold of FIGS. 20E-F. As illustrated in FIGS. 20E-F, the windows and door, which extruded 50 μm beyond the pineapple surface, and the leaf petals on top, were also replicated without loss of feature detail.

### Example 4

As illustrated in FIGS. 21 and 22A-E, experiments were conducted regarding the potential ability for toxic leachates from 3D prints to be transferred to PDMS using method of FIG. 1 and using a method of directly developing PDMS molds from the 3D-print. Additionally, UV and heat treatments were applied to a subset of the 3D prints before either direct molding or using the disclosed method, to see if any potential toxicity of the materials was eliminated. Treated media was then applied to monolayers of C2C12 mouse pre-myoblasts for 2 days, after which their metabolic activity was measured.

C2C12 mouse myoblast cells were maintained using DMEM+10% fetal bovine serum+1% penicillin/streptomycin+1% glutamax+1% non-essential amino acids. To test the potential toxicity of 3D printed materials, and their potential ability to leach into either PDMS directly molded on the print or PDMS replicas formed using the disclosed method, dog-bone molds were formed comprising of the following materials: clear resin printed 3D-prints, PDMS that was directly molded off of the clear resin, or PDMS that was replicated using the disclosed method. Similarly, these materials were created using clear resin that received an additional 30 minutes of UV (Sterilizer, 6W) and 30 minutes of heat (60° C.) treatment after printing. The PDMS was then directly molded or replicated using the disclosed method. This resulted in a total of 6 potentially toxic medias. These materials were soaked in the following media for 5 days and then sterile filtered: clear RPMI1640 +10% fetal bovine serum+1% penicillin/streptomycin+1% glutamax+1% non-essential amino acids. Additionally, two other medias were tested as positive and negative controls: the same clear RPMI formulation that was used for soaking the potentially toxic materials, with (positive control) and without (negative control) fetal bovine serum. C2C12 cells were plated at a density of 5 k cells/cm² and allowed to plate for 24 hours, after which toxic media was applied.

As illustrated in FIG. 21, media soaked directly with 3D-prints was highly toxic, killing almost all cells. PDMS that was directly molded off the 3D-print exhibited reduced toxicity, although cell viability was still about 50% less than in controls. Media soaked with PDMS produced using the disclosed method showed the lowest toxicity, and in many cases, cells cultured in media that was soaked in the PDMS molds using the disclosed method had similar viability to positive control cells. As illustrated in FIG. 21, post-cure UV and heat treatment did not reduce the toxicity of the 3D printed materials and the resulting media. These results highlight that creating PDMS molds from a 3D-print using a hydrogel intermediary mold produce non-toxic molds without the need for additional treatments of the 3D-print itself. FIGS. 22A-E illustrate representative brightfield images of C2C12 cells after 48 hours of treatment with control media or media soaked in PDMS made by direct molding of the 3D-print with PDMS or molding of the PDMS with the disclosed method.

As illustrated in FIGS. 22A and 22C, the PDMS mold developed directly from the 3D-print and the PDMS mold developed using the disclosed method display different levels of metabolic activity when no UV or heat treatment is given to the 3D-print before molding. FIG. 22A shows the PDMS mold developed directly from the 3D-print, having significantly less metabolic activity than the PDMS mold developed from the disclosed method (e.g., hydrogel intermediary mold) illustrated in FIG. 22C. The 3D prints of FIGS. 22B and 22D were given UV and heat treatments before PDMS molding. As illustrated in FIGS. 22B and 22D, the PDMS direct mold from the 3D-print, as illustrated in FIG. 22B, displays significantly less metabolic activity compared to the PDMS mold developed using the disclosed method (e.g., hydrogel intermediary mold) as illustrated in FIG. 22D. FIGS. 22C-D illustrate similar metabolic activity to the control in FIG. 22E, confirming that the disclosed hydrogel intermediary mold method produces PDMS molds with little to no toxicity and a high capability for sustaining metabolic activity of cells.

### Example 5

To demonstrate the potential of the disclosed method to easily create engineered tissues and its ability to eliminate the potential for toxic chemicals from the 3D print to be transferred the PDMS replica, PDMS dog-bone stencils were developed using the hydrogel intermediary mold method. The PDMS dog-bone stencils were utilized to form micro-heart muscle (pHM) from human Induced Pluripotent Stem Cell (hiPSC) derived cardiomyocytes. hiPSC-cargiomyocytes self-assemble, forming a spontaneously beating engineered cardiac tissue within the PDMS molds, that were imaged for calcium transients and action potentials and then fixed for sarcomere immunostaining on tissue day 15. “Wild Type C” (WTC) human induced pluripotent stem cells (iPSC) were modified via knock-in of a single copy of GCaMP6f into the AAVS1 “safe harbor locus.” iPSC were cultured at 37 degrees C. in Essential 8 media on 6-well plates coated with growth factor-reduced Matrigel. Once approximately 85% confluency was reached, the cells were passaged into new wells using Accutase. iPSC were differentiated into cardiomyocytes using small molecule manipulation of Wnt signaling.

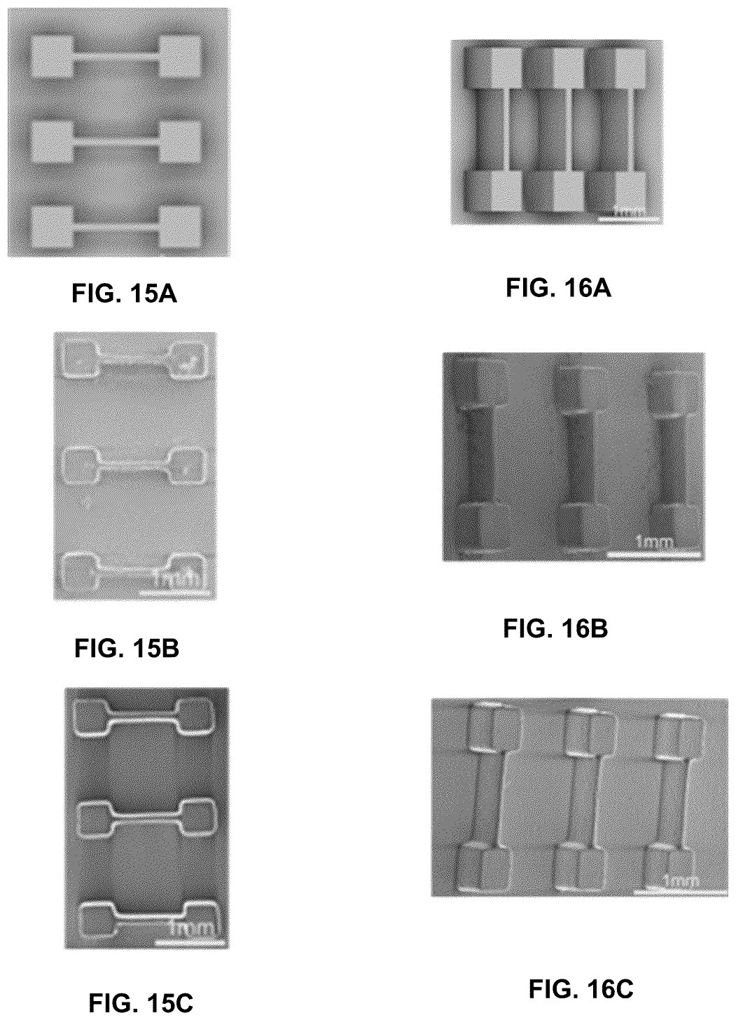

PDMS positive replicates of the 3D-printed molds were oxidized using air plasma for 90 seconds at high power. These were then treated with trichloro(1H,1H,2H,2H-perfluorooctyl) silane via vapor deposition to enable PDMS-off-PDMS molding for final stencil creation. Stencil molds with “dog-bone” shaped through-holes (e.g., similar to FIGS. 15A-16C) were formed by pouring Sylgard 184 over the treated PDMS molds, clamped between glass/acrylic plates and cured at 60 degrees C. for 4+ hours. This is then removed from the treated PDMS mold, resulting in a 1 mm thick sheet of PDMS containing dog-bone arrays. Each dog-bone shaped through-hole is 1 mm thick, forming the following: a tissue with 1 mm×1 mm knobs and a 400 μm×1 mm shaft.

To seed the PDMS stencils, they were cut, dipped in methanol, and placed into the wells of tissue culture well plates. The well plate was then placed into a 60 degrees C. oven overnight to reversibly bond the PDMS stencil to the well surface. The wells and stencils were then sterilized by soaking in 70% ethanol overnight, then coated with fibronectin to enable cardiomyocyte attachment to the substrate.

Un-purified iPSC-CM were singularized using 0.25% trypsin and seeded at a density of 70*10⁶ cells/mL at 3 μL per individual pHM (225,000 cells per tissue) without exogenous matrix. Seeded cells were incubated at 37 degrees C. for 60 minutes before adding media to limit cell loss, using DMEM with 20% FBS, 10 μM Y-27632, 150 μg/mL L-ascorbic acid, 4 μg/mL Vitamin B12, and 3.2 μg/mL penicillin. μHM typically began spontaneous beating within 24-48 hours of seeding; upon observing beating tissues the media was changed to RPMI/B-27, 150 μg/mL L-ascorbic acid, 4 μg/mL Vitamin B12, and 3.2 μg/mL penicillin (collectively called R+ media). μHM were then fed R+ media every 2-3 days until termination.

Tissues were fixed using increasing concentrations of paraformaldehyde from 1% A to 4%. PDMS stencils were then removed, and tissues were embedded in 1% agar dissolved in MilliQ water. The agar-embedded tissues were then cryoprotected using 15% and 30% sucrose, flash frozen in OCT and cryosectioned at 8-15 μm. Sectioning was done parallel to the tissue longitudinal axis. Samples were permeabilized with 0.1% Triton-X-100 for 20 minutes and blocked using 5% BSA in 0.1% Triton-X-100 for 45 minutes at room temperature. Sarcomeric α-actinin primary antibody (Sigma EA-53) was incubated overnight at 4 degrees C., secondary antibodies were incubated at room temperature for 2 hours, and cell nuclei (ThermoFisher Hoechst 33342) were stained at room temperature for 10 minutes. The samples were then protected against photobleaching with ProLong Gold (Invitrogen P36930) and imaged on a Nikon Eclipse Tsr2 inverted microscope equipped with a Hamamatsu ORCA 389 Flash 4.0 V3 digital CMOS camera and a Lumencor AURA light engine.

These cells were genetically modified to express GCaMP6f to image calcium handling dynamics, and Berst-1 voltage sensitive dye was used to visualize the action potential. Tissues were imaged on a Nikon Eclipse Tsr2 inverted microscope equipped with a Hamamatsu ORCA Flash 4.0 V3 digital CMOS camera and a Lumencor AURA light engine. A Tokai thermal plate was used to maintain μHM temperature during imaging. Videos were imaged at 200-450 fps. The MATLAB Bio-Formats package was used along with custom MATLAB software to plot waveforms.

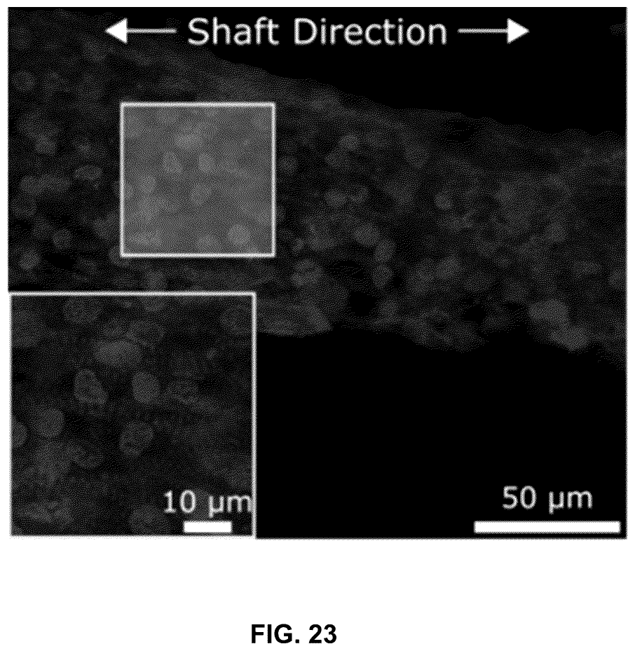

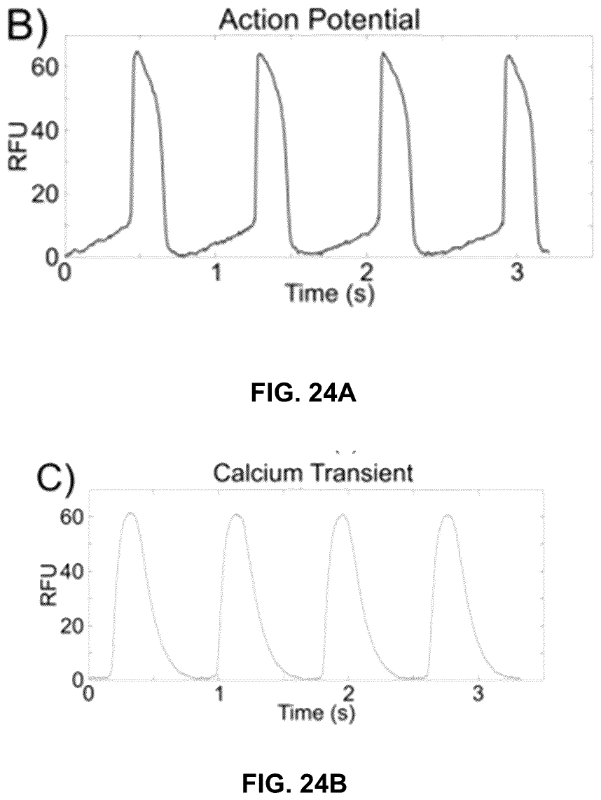

As illustrated in FIG. 23, the cardiomyocytes in these tissues express striated sarcomeric α-actinin and are aligned along the major tissue axis. As illustrated in FIG. 24A, these tissues exhibited robust action potential. As illustrated in FIG. 24B, the tissues exhibited calcium transient waveforms. These results highlight the biocompatibility of the PDMS mold developed using the hydrogel intermediary mold method.

### Example 6

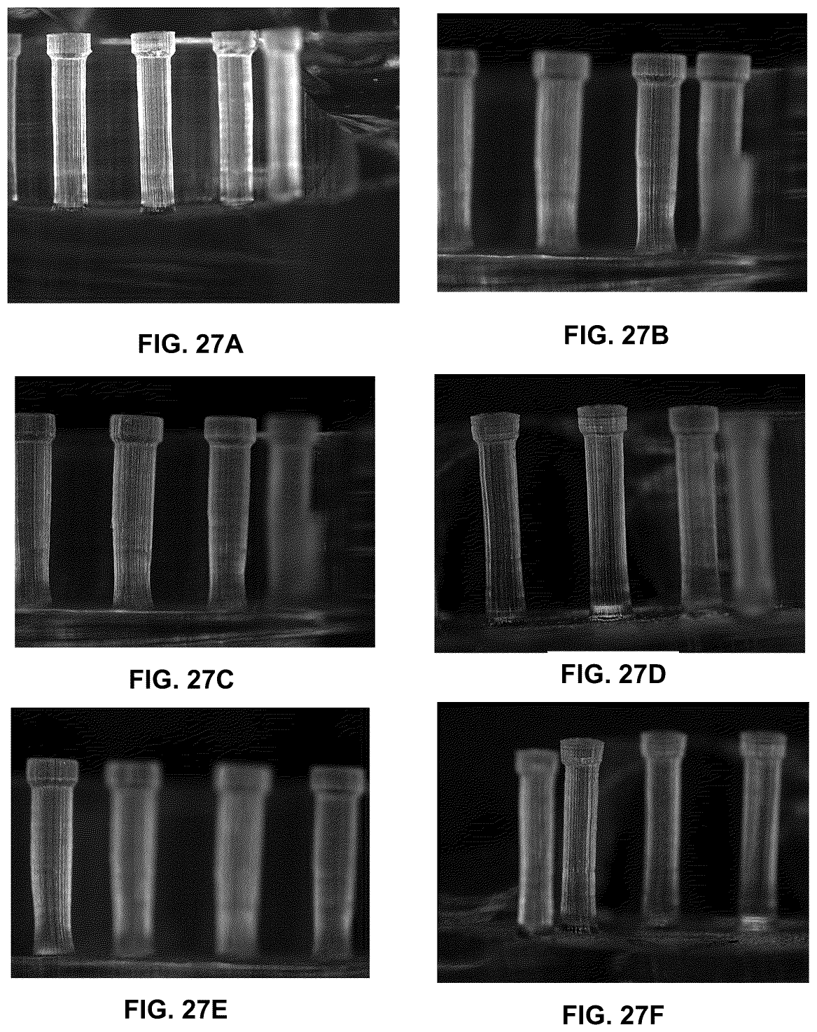

To further highlight the capabilities of the disclosed method, a PDMS mold for an 8 post micro-TUG (μTUG) design was formed, as illustrated in FIGS. 25 and 27A-F. The 8 post design was first 3D printed. The 8 post micro-tug design had a 3.26 mm diameter well with a 1.25 mm depth. Each post was designed with a 200 μm diameter with a 1.125 mm height. Each post had a top cap having a 300 μm diameter and a 125 μm height. The posts were designed to have a distance of 1.86 mm from the post directly across from each post.

The 3D printed 8 post micro-tug design was formed using clear resin. The prints were double washed for 15 minutes each with isopropyl alcohol, then air dried and post-cured under UV for 30 minutes. Supports on the underside of the 3D print were then removed.

1.5% wt/v agar in tap water was boiled. A laboratory detergent (e.g., Triton-X-100) was mixed to a final concentration of 0.2% during the time it took for the agar to cool below the glass transition temperature of the 3D print. After reaching 60 degrees C., the agar/laboratory detergent mixture was poured onto the 3D print and cooled down for 10 minutes in room temperature before being crosslinked over 20 minutes at 4 degrees C. Next, the 3D print was removed from the agar hydrogel, forming a negative agar hydrogel mold.

Sylgard 184 mixture (Dow Corning) was prepared by mixing the crosslinker and base with 1:10 ratio and degassed under vacuum chamber. In parallel, Sylgard 527 mixture (Dow) was prepared by mixing Part A and Part B with 1:1 ratio, then degassed in the same way as Syl-184. Afterwards, to make the compliant pillars, Sylgard 184 mixture was mixed with Sylgard 527 mixture with ratio 1:4, respectively, to yield PDMS with an elastic modulus of 114 kPa; this PDMS mixture was poured into the agar mold, degassed and kept at 37 degrees C. in an oven for 2 hours to let the PDMS partially crosslink on the pillars. Afterwards, the remaining agar mold was filled with the remaining Sylgard 184 (to yield rigid wells) until the thickness of the whole platform reached about 3-4 mm, then kept in the 37 degrees C. overnight. The next day, devices were removed from agar and underwent final crosslinking at 60 degrees C. overnight. This process allowed us to reproducibly form compliant, 114 kPa pillars within more rigid (2 MPa) microwells and devices that could easily be handled by end users. As illustrated in FIGS. 25 and 27A-F, the resulting PDMS mold displays high fidelity to the original complex 3D print.

The particular variations disclosed above are illustrative only, as the variations may be modified and practiced in different but equivalent manners apparent to those skilled in the art having the benefit of the teachings herein. It is therefore evident that the particular variations disclosed above may be altered or modified, and all such variations are considered within the scope and spirit of the application. Accordingly, the protection sought herein is as set forth in the description. Although the present variations are shown above, they are not limited to just these variations, but are amenable to various changes and modifications without departing from the spirit thereof. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed variations teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method of making a mold, the method comprising:
printing a 3D printed resin mold;
casting a hydrogel over the 3D printed resin mold to create a crosslinked hydrogel negative mold, wherein the hydrogel crosslinks in a manner that is not inhibited by small molecules released from the 3D printed resin mold and a surface of the 3D-printed resin mold; and
casting a silicone rubber elastomer over the hydrogel negative mold to create a master mold.

2. The method of claim 1, wherein the silicone rubber elastomer is poly(dimethyl siloxane).

3. The method of claim 1, wherein the crosslinked hydrogel negative mold comprises a high crosslink density.

4. The method of claim 3, wherein the crosslink density is greater than about 2.5 mol/cm³.

5. The method of claim 4, wherein the crosslink density is greater than about 4 mol/cm³.

6. The method of claim 1, wherein the crosslinked hydrogel negative mold has a toughness of greater than 8 kJ/³.

7. The method of claim 1, wherein the hydrogel is agar, alginate, hyaluron, or gelatin.

8. The method of claim 7, wherein the hydrogel comprises 1.5% w/v agar.

9. The method of claim 7, wherein the hydrogel comprises 1% high molecular weight alginate and 2% low molecular weight alginate.

10. The method of claim 1, wherein the master mold is a 1:1 replication of the 3D printed resin mold.

11. The method of claim 10, wherein the master mold replicates features as small as 10 μm of the 3D printed resin mold.

12. The method of claim 1, wherein the master mold does not contain any leachate from the 3D printed resin mold.

13. The method of claim 1, wherein the master mold is biocompatible and does not contain any toxins that would inhibit cell growth.

14. A master mold made using the method of claim 1.

15. A master mold comprising a silicone rubber elastomer, wherein the master mold has a Procrustes score of greater than 0.99 when compared to a 3D printed resin mold used to make the master mold, and wherein the master mold does not contain any leachate from the 3D printed resin mold.

16. A method of forming an engineered tissue, the method comprising:
printing a 3D printed mold;
casting a hydrogel over the 3D printed mold to create a crosslinked hydrogel negative mold;
casting a silicone rubber elastomer over the crosslinked hydrogel negative mold to create a master mold; and
seeding the master mold with cells to produce the engineered tissue.

17. The method of claim 16, wherein the engineered tissue comprises cardiomyocytes and stromal cells.

18. The method of claim 17, wherein the engineered tissue displays an action potential and a calcium transient.

19. The method of claim 16, wherein the engineered tissue is a model of an in vivo organ system.

20. The method of claim 16, wherein the master mold is placed in a tissue culture well plate prior to being seeded with cells.
